SEQUENCE LISTING (5 sequences)

SEQ ID NO: 1 - DNA, 1368 bp, Homo sapiens
atggatcccgggcagcagccgccgcctcaaccggccccccagggccaagggcagccgccttcgcagcccccgcaggggcagggcccgccgtccggacccgggcaaccggcacccgcggcgacccaggcggcgccgcaggcaccccccgccgggcatcagatcgtgcacgtccgcggggactcggagaccgacctggaggcgctcttcaacgccgtcatgaaccccaagacggccaacgtgccccagaccgtgcccatgaggctccggaagctgcccgactccttcttcaagccgccggagcccaaatcccactcccgacaggccagtactgatgcaggcactgcaggagccctgactccacagcatgttcgagctcattcctctccagcttctctgcagttgggagctgtttctcctgggacactgacccccactggagtagtctctggcccagcagctacacccacagctcagcatcttcgacagtcttcttttgagatacctgatgatgtacctctgccagcaggttgggagatggcaaagacatcttctggtcagagatacttcttaaatcacatcgatcagacaacaacatggcaggaccccaggaaggccatgctgtcccagatgaacgtcacagcccccaccagtccaccagtgcagcagaatatgatgaactcggcttcagccatgaaccagagaatcagtcagagtgctccagtgaaacagccaccacccctggctccccagagcccacagggaggcgtcatgggtggcagcaactccaaccagcagcaacagatgcgactgcagcaactgcagatggagaaggagaggctgcggctgaaacagcaagaactgcttcggcaggtgaggccacaggagttagccctgcgtagccagttaccaacactggagcaggatggtgggactcaaaatccagtgtcttctcccgggatgtctcaggaattgagaacaatgacgaccaatagctcagatcctttccttaacagtggcacctatcactctcgagatgagagtacagacagtggactaagcatgagcagctacagtgtccctcgaaccccagatgacttcctgaacagtgtggatgagatggatacaggtgatactatcaaccaaagcaccctgccctcacagcagaaccgtttcccagactaccttgaagccattcctgggacaaatgtggaccttggaacactggaaggagatggaatgaacatagaaggagaggagctgatgccaagtctgcaggaagctttgagttctgacatccttaatgacatggagtctgttttggctgccaccaagctagataaagaaagctttcttacatggttataggcg

SEQ ID NO: 2 - DNA, 1080 bp, Mus musculus
atgaaccccaagcccagctcatggcggaaaaagatcctcccggagtccttctttaaggagcccgattccggctcgcactcgcgccaatccagcacagactcatcaggcggccacccggggcctcgactagctggcggcgcgcagcacgtccgctcgcactcgtcgcccgcatccctgcagctgggcaccggtgcgggagccgctggaggccctgcacagcagcatgcacatctccgccagcagtcctatgacgtgaccgacgagctgccgttgccccccgggtgggagatgaccttcacggccactggccagagatacttccttaatcacatagagaaaatcaccacatggcaagaccccaggaaggtgatgaatcagcctctgaatcatgtgaacctccacccgtccatcacttccacctcggtgccacagaggtccatggcagtgtcccagccgaatctcgcaatgaatcaccaacaccagcaagtcgtggccactagcctgagtccacagaaccacccgactcagaaccaacccacagggctcatgagtgtgcccaatgcactgaccactcagcagcagcagcagcagaaactgcggcttcagaggatccagatggagagagagaggattaggatgcgtcaagaggagctcatgaggcaggaagctgccctctgccgacagctccccatggaaaccgagaccatggcccctgtcaacacgcctgccatgagcacagatatgagatctgtcaccaacagtagctcagatcctttcctcaatggagggccctatcattcacgggagcagagcacagacagtggcctggggttagggtgctacagtgtccccacaactccagaagacttcctcagcaacatggacgagatggatacaggtgaaaattccggtcagacacccatgaccgtcaatccccagcagacccgcttccctgatttcctggactgccttccaggaacaaatgttgacctcgggactttggagtctgaagatctgatccctctcttcaatgatgtaagttgtctgaacaaaagcgagccctttctaacctggctgtaa

SEQ ID NO: 3 - DNA, 1515 bp, Homo sapiens
atggatcccgggcagcagccgccgcctcaaccggccccccagggccaagggcagccgccttcgcagcccccgcaggggcagggcccgccgtccggacccgggcaaccggcacccgcggcgacccaggcggcgccgcaggcaccccccgccgggcatcagatcgtgcacgtccgcggggacgcggagaccgacctggaggcgctcttcaacgccgtcatgaaccccaagacggccaacgtgccccagaccgtgcccatgaggctccggaagctgcccgactccttcttcaagccgccggagcccaaatcccactcccgacaggccgctactgatgcaggcactgcaggagccctgactccacagcatgttcgagctcatgcctctccagcttctctgcagttgggagctgtttctcctgggacactgacccccactggagtagtctctggcccagcagctacacccacagctcagcatcttcgacagtctgcttttgagatacctgatgatgtacctctgccagcaggttgggagatggcaaaaaccagcagcgggcaacgttattttctcaatcacatcgatcagacaacaacatggcaggaccccaggaaggccatgctgtcccagatgaacgtcacagcccccaccagtccaccagtgcagcagaatatgatgaactcggcttcaggtcctcttcctgatggatgggaacaagccatgactcaggatggagaaatttactatataaaccataagaacaagaccacctcttggctagacccaaggcttgaccctcgttttgccatgaaccagagaatcagtcagagtgctccagtgaaacagccaccacccctggctccccagagcccacagggaggcgtcatgggtggcagcaactccaaccagcagcaacagatgcgactgcagcaactgcagatggagaaggagaggctgcggctgaaacagcaagaactgcttcggcaggcaatgcggaatatcaatcccagcacagcaaattctccaaaatgtcaggagttagccctgcgtagccagttaccaacactggagcaggatggtgggactcaaaatccagtgtcttctcccgggatgtctcaggaattgagaacaatgacgaccaatagctcagatcctttccttaacagtggcacctatcactctcgagatgaggctacagacagtggactaagcatgagcagctacagtgtccctcgaaccccagatgacttcctgaacagtgtggatgagatggatacaggtgatactatcaaccaaagcaccctgccctcacagcagaaccgtttcccagactaccttgaagccattcctgggacaaatgtggaccttggaacactggaaggagatggaatgaacatagaaggagaggagctgatgccaagtctgcaggaagctttgagttctgacatccttaatgacatggagtctgttttggctgccaccaagctagataaagaaagctttcttacatggttatag

SEQ ID NO: 4 - DNA, 1083 bp, Mus musculus
atgaaccccaagcccagctcatggcggaaaaagatcctcccggagtccttctttaaggagcccgattccggctcgcactcgcgccaatccagcacagactcatcaggcggccacccggggcctcgactagctggcggcgcgcagcacgtccgctcgcactcgtcgcccgcatccctgcagctgggcaccggtgcgggagccgctggaggccctgcacagcagcatgcacatctccgccagcagtcctatgacgtgaccgacgagctgccgttgccccccgggtgggagatgaccttcacggccactggccagagatacttccttaatcacatagagaaaatcaccacatggcaagaccccaggaaggtgatgaatcagcctctgaatcatgtgaacctccacccgtccatcacttccacctcggtgccacagaggtccatggcagtgtcccagccgaatctcgcaatgaatcaccaacaccagcaagtcgtggccactagcctgagtccacagaaccacccgactcagaaccaacccacagggctcatgagtgtgcccaatgcactgaccactcagcagcagcagcagcagaaactgcggcttcagaggatccagatggagagagagaggattaggatgcgtcaagaggagctcatgaggcaggaagctgccctctgccgacagctccccatggaaaccgagaccatggcccctgtcaacacgcctgccatgagcacagatatgagatctgtcaccaacagtagctcagatcctttcctcaatggagggccctatcattcacgggagcagagcacagacagtggcctggggttagggtgctacagtgtccccacaactccagaagacttcctcagcaacatggacgagatggatacaggtgaaaattccggtcagacacccatgaccgtcaatccccagcagacccgcttccctgatttcctggactgccttccaggaacaaatgttgacctcgggactttggagtctgaagatctgatccctctcttcaatgatgtagagtctgttctgaacaaaagcgagccctttctaacctggctgtaa

SEQ ID NO: 5 - DNA, 413 bp, Artificial
  tet-On promoter
gtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgccacc

(19) United States
(12) Patent Application Publication
(10) Pub. No.: US 2018/0245038 A1
(43) Pub. Date: Aug. 30, 2018

(54) METHOD FOR GENERATING SOMATIC STEM CELLS

(71) Applicant: UNIVERSITA' DEGLI STUDI DI PADOVA, Padova (IT)

(72) Inventors: Stefano PICCOLO, Padova (IT); Luca AZZOLIN, Padova (IT); Tito PANCIERA, Padova (IT); Michelangelo CORDENONSI, Vigodarzere (IT); Francesca ZANCONATO, Vicenza (IT); Atsushi FUJIMURA, Kumamoto (JP)

(21) Appl. No.: 15/757,585

(22) PCT Filed: Sep. 4, 2015
(86) PCT No.: PCT/EP2015/070305

§ 371 Date: Mar. 5, 2018

(51) Int. Cl. C12N 5/074; C12N 5/0797; C12N 5/071; C07K 14/47; C12N 9/10; A61K 35/28

(52) U.S. Cl. CPC C12N 5/0607; C12N 5/0623; C12N 5/0678; C07K 14/4705; C12N 9/1025; A61K 38/00; C12N 2506/095; C12N 2506/08; C12N 2506/22; C12N 2510/00; A61K 35/28

(57) ABSTRACT

The present invention provides a method for generating somatic stem cells out of differentiated cells, somatic stem cells obtained by this method and a vector or composition for use in this method.

### FIELD OF THE INVENTION

The present invention relates generally to a method for generating somatic stem cells and to somatic stem cells generated by said method. The invention further related to a vector, a composition and a kit for use in said method, for use in regenerative medicine, tissue repair, ex-vivo or in vivo modeling of human diseases, such as cancer, liver failure, diabetes, neurological deficiencies.

### BACKGROUND

Stem cells (SCs) display the capacity to renew themselves when they divide, and to generate a differentiated progeny. Somatic SCs operate in multiple adult organs for continuous tissue renewal or repair after injury. Yet, these cells are still mainly defined by operational definitions and cell surface markers rather than the molecular traits that govern their special status (Fuchs, E. & Chen, T. A matter of life and death: self-renewal in stem cells. EMBO reports 14, 39-48 (2013)). Unlimited availability of normal, somatic SCs will be critical for effective organ repopulation in regenerative medicine applications, to understand SC biology and for disease modeling in the Petri dish. These efforts are frustrated by the fact that SCs are rare and difficult to purify from native tissues or to expand ex vivo. A recent important step forward in this direction has been the description of culture systems allowing adult epithelial SCs of endodermal origin to expand and self-organize into “organoids” (Sato, T. & Clevers, H. Growing self-organizing mini-guts from a single intestinal stem cell: mechanism and applications. Science 340, 1190-1194 (2013)). Yet, these methods still require the isolation of native SCs as starting material.

Direct conversion of terminally differentiated cells back into their corresponding tissue-specific SCs may represent an attractive alternative to obtain somatic SCs. Indeed, several reports have recently highlighted a surprising plasticity in somatic cell fates, as differentiated cells can return to a SC status under special conditions, such as tissue damage (Blanpain, C. & Fuchs, E. Stem cell plasticity. Plasticity of epithelial stem cells in tissue regeneration. Science 344, 1242281 (2014); and Tetteh, P. W., Farin, H. F. & Clevers, H. Plasticity within stem cell hierarchies in mammalian epithelia. Trends in cell biology (2014)). However, the identity of the factors able to control the somatic SC status remains poorly understood, limiting the exploitation of such plasticity.

YAP (Yes-associated protein) and its paralog TAZ (transcriptional co-activator with PDZ-binding motif) are the main downstream effectors of the Hippo signaling pathway. This pathway is an evolutionally conserved signal cascade, which plays pivotal roles in organ size control and tumorigenesis from Drosophila to mammals (Guo, L and Teng, L, Int J Oncol. 2015 April; 46(4):1444-52.).

Possible roles of these pathways in direct conversion of terminally differentiated cells back into their corresponding tissue-specific SCs still remain elusive.

Evaluation of methods for generating somatic stem cells and searching for factors involved in the underlying molecular mechanisms continue.

### SUMMARY OF THE INVENTION

The present invention provides a method for generating somatic stem cells and a somatic stem cell obtained by said method. The present invention further provides a vector, a composition and a kit for use in the method of the invention.

In one aspect, the present invention provides a method for generating somatic stem cells, comprising the steps of:
a. providing at least one differentiated cell, committed progenitor or partially differentiated cell;
b. inducing an increased expression or activity of a YAP protein, and/or a TAZ protein, and/or a functional fragment of the YAP and/or the TAZ protein, and/or an activated version of the YAP and/or the TAZ protein, or derivatives thereof in at least one differentiated cell or committed progenitor or partially differentiated cell;
c. generating a somatic stem cell out of said differentiated cell, committed progenitor or partially differentiated cell.

In some embodiments, the expression of the YAP/TAZ protein and/or the functional fragment of the YAP/TAZ and/or the activated version of the YAP/TAZ protein, or derivatives thereof in the at least one differentiated cell or committed progenitor or partially differentiated cell may be increased transiently. This improves the security of the invention, since the induced over expression of YAP/TAZ protein may be stopped once the somatic stem cell has been generated.

In some embodiments, said YAP/TAZ protein may be endogenous.

The activity of endogenous YAP/TAZ protein may be increased by influencing a biological activity of the endogenous YAP/TAZ protein, and/or by influencing a cellular stability of the endogenous YAP/TAZ protein, and/or by a influencing a cellular localization of the endogenous YAP/TAZ protein.

According to one embodiment, this may be done by applying to the at least one differentiated cell or committed progenitor or partially differentiated cell a composition comprising a substance for influencing the biological activity of the endogenous YAP/TAZ protein, and/or for influencing a cellular stability of the endogenous YAP/TAZ protein, and/or for influencing a cellular localization of the endogenous YAP/TAZ protein.

In accordance with one embodiment, the method may comprise the step of transfecting the at least one differentiated cell or committed progenitor or partially differentiated cell of step a) with a vector comprising a nucleotide sequence coding for a protein which induces the increased expression or activity of the endogenous YAP/TAZ protein.

According to some embodiments of the present invention, the increased expression of the YAP/TAZ protein and/or the functional fragment and or/said activated version, and/or derivatives thereof in the at least one differentiated cell or committed progenitor or partially differentiated cell may be ectopic. The method may then further comprise the step of transfecting the at least one differentiated cell of step a) with a vector comprising a nucleotide sequence coding for a wild-type YAP protein and/or a nucleotide sequence coding for a wild-type TAZ protein and/or a nucleotide sequence coding for a functional fragment of the wild-type YAP protein and/or the wild-type TAZ protein, and/or a nucleotide sequence coding for the activated version, and/or derivatives thereof.

The transfection of the at least one differentiated cell may be performed using a lentiviral vector. This allows for infection of non-dividing cells. Further, the vector can be integrated into the genome of the differentiated cell.

In accordance with one embodiment of the present invention, expression of the wild-type YAP/TAZ protein and/or the functional fragment of the YAP/TAZ protein and/or the activated version of the YAP/TAZ protein, and/or derivatives thereof is under the control of an inducible promoter. An example for such inducible promoter is a doxycyclin-inducible promoter. Transient expression may thereby be provided by the use of self-inactivating lentiviral vectors (in which the transgene may be deleted from the receiving cell genome) or by adenoviral vectors (that never integrate in the host genome) in order to improve the security of the method.

In the above method, the starting cell can be any mammalian cell, including, but not limited to, terminally differentiated cells. In some embodiments, the cell is a human cell, mouse cell, or rat cell. Examples of differentiated cells include, e.g., differentiated mammary gland cells, differentiated neural cells and differentiated pancreatic cells. The cell may be a terminal differentiated cell, a committed progenitor or a partially differentiated cell or a cell with dual stem-differentiated traits.

According to one embodiment, the step of generating a somatic stem cell comprises verifying at least one characteristic typical for somatic stem cells. For example, morphological characteristics of the cells may be used to check whether somatic stem cells have been generated. Alternatively or additionally, on a molecular level, it may be tested whether typical SC markers are detectable on the cell after executing step b) of the above method.

According to one embodiment, in order to verify the generation of a somatic stem cell, self renewal potential of the cell may be tested.

Alternatively or additionally, if the differentiated cells are differentiated mammary gland cells, the ability to self organize into mammary tissue like structures may be tested.

Further, multilineage differentiation ability of the cells may be tested in order to verify the generation of a somatic stem cell.

According to one embodiment, endogenous YAP/TAZ expression may be measured in the at least one differentiated cell or committed progenitor or partially differentiated cell after having stopped the induced increased expression of the ectopic YAP protein, and/or the TAZ protein, and/or the functional fragment of the YAP and/or the TAZ protein, and/or an activated version of the YAP and/or the TAZ protein, or derivatives thereof, in the at least one differentiated cell according to step b). Reactivation of endogenous YAP/TAZ expression may indicate the generation of somatic stem cells.

According to one embodiment, endogenous YAP/TAZ expression may be measured in the at least one differentiated cell or committed progenitor or partially differentiated cell after having stopped influencing a biological activity, a cellular stability or a cellular localization of an endogenous YAP/TAZ protein. Reactivation of endogenous YAP/TAZ expression after suspension of external activation may indicate the generation of somatic stem cells.

According to one embodiment, the step of generating a somatic stem cell comprises verifying the loss of expression of terminal differentiation markers of the cell after implementing step b) of the above method. Further, expression of typical SC markers may be measured.

Methods suitable to determine whether expression of YAP/TAZ or their biologically active derivative has reprogrammed a somatic cell into a stem cell include expression studies by means of polyacrylamide gel electrophoresis and related blotting techniques such as western blot paired with chromogenic or fluorescence and luminescence-based detection procedures; it also include immunofluorescence in cellular specimens aimed to determine acquired expression of genes typical of somatic SCs of a given tissue. Gene expression (i.e. downregulation of differentiated markers and upregulation of SC-markers) may be demonstrated by in situ hybridization and PCR-based procedure such as qPCR, RT-PCR, qRT-PCR, RT-qPCR, Light Cycler®, TaqMan® Platform and Assays, Northern blot, dot blot, microarrays, next generation sequencing (VanGuilder, Biotechniques (2008), 44: 619-26; Elvidge, Pharmacogenomics (2006), 7: 123-134; Metzker, Nat Rev Genet (2010), 11: 31-46). The corresponding experimental conditions are also established according to conventional protocols described, for example, in Sambrook, Russell “Molecular Cloning, A Laboratory Manual”, Cold Spring Harbor Laboratory, N.Y. (2001); Ausubel, “Current Protocols in Molecular Biology”, Green Publishing Associates and Wiley Interscience, N.Y. (1989), or Higgins and Hames (Eds.) “Nucleic acid hybridization, a practical approach” IRL Press Oxford, Washington D.C., (1985). The setting of conditions is well within the skill of the artisan and can be determined according to protocols described in the art.

In addition to gene expression, the acquisition of a somatic SC fate can be measured by functional assays, in particular the acquisition of proliferative properties and ability of the induced/reprogrammed cell to be serially passaged and expanded, while retaining the ability to generate a differentiated progeny. Somatic SC acquisition can be also validated by the ability to regenerate tissues in animal models.

In another aspect, the present invention provides a somatic stem cell, obtained by anyone of the methods described above.

The induced somatic stem cell according to the present invention may be used in a regenerative medicine application. For example, the somatic stem cells may be used for generating tissues for transplantation. The somatic stem cells may be used to repair or replace tissue or organ function lost due to age, disease, organ damage, or congenital defects. The induced somatic stem cells may then be used to generate cells and tissue ex-vivo, to correct genetic defects, to expand or generate de novo stem cells in vivo, including self-propagating cells with augmented properties in comparison with natural/endogenous stem cells.

In a further aspect of the present invention, it is provided a vector comprising a nucleotide sequence coding for a wild-type YAP protein, and/or a nucleotide sequence coding for a wild-type TAZ protein, and/or a nucleotide sequence coding for a functional fragment of said YAP and/or said TAZ protein, and/or a nucleotide sequence coding for an activated version of said YAP and/or said TAZ protein, and/or a nucleotide sequence coding for a protein which induces an increased expression or activity of an endogenous YAP/TAZ protein, or derivatives thereof, wherein the transcription of said nucleotide sequence is under the control of an inducible promoter, for use in any one of the methods according to the present invention.

According to one embodiment, the nucleotide sequence may comprise anyone of the sequences Seq ID No. 1, Seq ID No. 2, Seq ID No. 3, or Seq ID No 4.

The vector may further comprise the nucleotide sequence according to Seq ID No. 5.

In a further aspect of the present invention, it is provided a composition comprising a substance for influencing a biological activity of an endogenous YAP/TAZ protein, and/or for influencing a cellular stability of said endogenous YAP/TAZ protein, and/or for influencing a cellular localization of said endogenous YAP/TAZ protein, for use in any one of the methods according to the present invention.

In a further aspect of the present invention, it is provided a kit, comprising a vector according to the present invention and/or comprising a composition in accordance with the present invention.

According to one embodiment, the kit may include a vector and/or a composition being prepared to be administered orally, rectally, by injection, inhalation, or topically.

### BRIEF DESCRIPTION OF THE DRAWINGS

FIG. 1 shows how YAP and TAZ convert luminal differentiated cells in yMaSCs, wherein

FIG. 1a shows a FACS profile of the distribution of Lin−/EpCAM+ mammary cells according to their CD49f/CD61 antigenic profile;

FIG. 1b shows Western blots for YAP, TAZ and p63; GAPDH serves as loading control;

FIG. 1c shows qRT-PCRs for Ctgf and Ax1 in the indicated cell populations (mean+s.d). Results are representative of three independent experiments (each using mammary glands from n=20 mice), performed in triplicate;

FIG. 1d shows a schematic representation of the experiments performed with LD cells. Doxy stands for doxycycline; and

FIGS. 1e-f show representative images (e) and quantifications (f) of mammary colonies formed by the indicated cells, 15 days after seeding in mammary colony medium. Data in (f) are presented as mean+s.d. and are representative of five independent experiments, each with six technical replicates.

FIG. 2 shows that yMaSCs display mammary gland reconstitution ability, wherein

FIG. 2a shows representative images of yMaSCs outgrowths at the indicated time points. Until day 14, cultures were in mammary colony medium. After transfer to organoid conditions (see scheme in FIG. 1d), cells were maintained and passaged without doxycycline. Scale bar, 250 μm;

FIG. 2b shows anti-YFP immunostaining of the lineage tracing experiment showing that yMaSC-derived colonies and organoids originate from tamoxifen-treated K8-CreERT2; R26-LSL-YFP LD cells. Scale bars, 49 μm;

FIG. 2c-f show organoids from MaSCs and yMaSCs (from wtYAP) expressed basal/stem (α-Sma, K14, p63) and luminal markers (K8, K19, scale bars in IF pictures is 17 μm) and β-casein (qRT-PCR) when treated with prolactin. In f, data were normalized to Gapdh expression and presented as mean+s.d.; results are representative of two independent experiments performed in triplicate;

FIG. 2g show unsupervised hierarchical clustering of differentially expressed genes between LD cells, organoids from MaSCs (M) and organoids from yMaSCs (yM). Each column represents one separated biological sample. Only probe sets with a coefficient of variation larger than the 90^{th} percentile of the coefficients of variation in the entire dataset were considered for clustering. Genes are ordered according to the decreasing average expression level in LD cells;

FIGS. 2h-j show the mammary gland in vivo outgrowths generated by stable GFP-expressing yMaSCs (from wtYAP) in virgin females. h: whole-mount images (left, native GFP fluorescence; right, hematoxylin staining). i: histological section. j: representative sections stained for GFP and the indicated markers; and

FIG. 2 k-l show mammary gland reconstitution generated by single-cell derived yMaSC organoids in a impregnated female. k: whole-mount images (left, native GFP fluorescence; right, hematoxylin staining). l: histological section. Note that upon gestation and lactation, the mammary gland is constituted by alveoli filled with milk.

FIG. 3 shows how YAP and TAZ convert neurons in yNSCs, wherein

FIG. 3a-b show Representative confocal images of NSCs (plated as monolayer) and neurons, costained for YAP/Nestin and YAP/TuJ1, respectively. Nuclei were stained with DAPI. Scale bar: 23 μm;

FIG. 3c shows a Schematic representation of the experiments performed with hippocampal or cortical neurons;

FIG. 3d-f show Representative images of yNSCs neurospheres (second passage, P2) derived from hippocampal (d) or cortical (e) neurons. Images from negative control transduced neurons are shown as reference (d, e). Neurospheres from endogenous NSCs are presented as comparison (f). Scale bars, 210 μm;

FIG. 3g shows how P1 yNSCs were dissociated to single cells and replated at clonal density for neurosphere formation in the absence of doxycycline for further passages (P2, P3, P4). NSCs are presented as comparison. Graphs are quantifications of neurospheres formed by the indicated cells. Results are representative of at least 8 (P2), 6 (P3) and 3 (P4) independent experiments performed in six replicates. Data are presented as mean+s.d.

FIG. 3h shows lineage tracing experiment showing that yNSCs originate from neurons. Panels are X-gal stainings for neurons (scale bar, 10 μm) from Thy1-Cre; R26-LSL-LacZmice and derived yNSCs (scale bars, 210 μm) at successive passages. Neurospheres from Thy1-Cre; R26-LSL-LacZ NSCs (scale bar 210 μm) are presented as negative control. See scheme in Extended Data FIG. 6a;

FIG. 3i shows Immunofluorescence for the indicated markers (scale bar, 23 μm) in neurons and established yNSCs plated as monolayer. Endogenous NSCs serve as positive control;

FIG. 3j shows unsupervised hierarchical clustering of differentially expressed genes between cortical neurons, yNSCs and NSCs. Each column represents one separated biological sample. Only probe sets with a coefficient of variation larger than the 90^{th} percentile of the coefficients of variation in the entire dataset were considered for clustering. Genes are ordered according to the decreasing average expression level in neurons; and

FIG. 3k-m show wtYAP-induced yNSCs, and endogenous NSCs as positive control, were plated and differentiated toward an astrocytic, a neuronal or an oligodendrocytic fate (see Methods). Panels represent confocal images for astrocytic marker GFAP (k), neuronal differentiation marker Tuj1 (l) and oligodendrocytic marker CNPase (m). Results are representative of three independent experiments performed in triplicate. Scale bars, 50 μm.

FIG. 4 shows how YAP converts pancreatic acinar cells to duct-like organoids; wherein

FIG. 4 a-b show representative images of a pancreatic duct fragment growing in pancreatic organoid medium at the indicated times, and after four passages in fresh Matrigel(b). Pictures are representative of three independent experiments performed with four technical replicates. Scale bars in a and b, 290 μm;

FIG. 4c-d, show serial images of a single acinar cell derived from R26-rtTA; tetO-YAP^{(S127A)} growing as cyst-like organoids at the indicated time points after Doxy addition (c) and after four passages in fresh Matrigel in the absence of Doxy (d). Pictures are representative of five independent experiments, performed with four technical replicates. Scale bars, 70 μm in c; 290 μm in d;

FIG. 4e-f show lineage-tracing experiments using the Ptf1a-CreERTM driver. See also Extended Data FIG. 9a for a scheme of the experiment. Panels are bright field and GFP-fluorescence pictures of transgenic YAP-expressing exocrine cells, at the indicated time points of Doxy treatment (e) and after passaging in absence of Doxy (f). The same acinar cells formed no organoidsin absence of doxycycline (Extended Data FIG. 9b). Scale bars, 70 μm in e; 130 μm in f;

FIG. 4g shows organoids from duct fragments (Ducts, bottom panels, as in (b)) and YAP-induced organoids (yDucts, middle panels) expressed the ductal marker SOX9 and were negative for the exocrine marker Amylase (data not shown), by immunofluorescence. Acinar cells (top panel) are shown as control. Scale bar, 80 μm; and

FIG. 4h shows unsupervised hierarchical clustering of differentially expressed genes between acini, yDucts and Ducts. Each column represents one separated biological sample. Only probe sets with a coefficient of variation larger than the 90^{th} percentile of the coefficients of variation in the entire dataset were considered for clustering. Genes are ordered according to the decreasing average expression level in acini.

FIG. 5 shows the characterization of FACS-sorted mammary cells This refers to FIG. 1a-c of the main text.

FIG. 5a, shows a FACS profile for EpCAM of the experiments represented in FIG. 1a;

FIG. 5b-c show qRT-PCRs and western blots for the indicated basal/stem and luminal markers in MaSCs, LP, and LD cells obtained by FACS. In b, data are normalized to Gapdh expression and are referred to MaSC levels for basal genes, to LP levels for Hey1, and to LD levels for all the other luminal markers (each set to 1). Results are representative of at least three independent experiments (each using mammary glands from n=20 mice) performed in triplicate. In c, GAPDH serves as loading control.

FIG. 5d shows representative images of mammary colonies formed by the indicated cells, growing at the indicated time points in mammary colony medium. MaSCs formed solid outgrowths, while LD remained as single cells. LP cells, despite being able to form acinar (cavitated) colonies, were unable to self-renew after passaging, or form organoids when transferred in 100% Matrigel/mammary organoid medium culture system (not shown). Pictures are representative of three independent experiments performed with six technical replicates. Scale bar, 170 μm.

FIG. 5e shows representative images of whole mount hematoxylin staining of cleared fat pads injected with purified MaSCs (leading to outgrowth of a ductal mammary tree) or LD cells as negative control. LP cells were similarly void of regenerative potential in vivo (not shown). Scale bars, 1 cm; and

FIG. 5f shows representative images of 3D colonies from wild-type (wt) or Yap^{fl/fl}; Taz^{fl/fl} MaSCs transduced with Ad-Cre or Ad-GFP as control. Scale bar, 250 μm.

FIG. 6 shows induction of MaSC traits in luminal differentiated cells by Y YAP/TAZ; wherein

FIG. 6a-b show primary mammary colonies from MaSCs and yMaSCs as in FIG. 1e,f were dissociated and re-seeded in mammary colony medium without doxycycline. Secondary colonies were counted 2 weeks after seeding and immediately dissociated and re-seeded in the same conditions for tertiary colonies formation. Graphs are quantifications of secondary (a) and tertiary (b) colonies formed by the indicated cells. Data are representative of two independent experiments performed with six technical replicates, and presented as mean+s.d.; and

FIG. 6c, shows detailed quantification of single LD cells in 96-well plates, reprogrammed to a MaSC-like state upon inducible YAP expression. LD cells expressing inducible EGFP or YAPS94A didn't form any colony.

FIG. 7 shows Characterization of mammary organoids derived from aSCs and yMaSCS. This refers to FIG. 2a-g.

FIG. 7a shows qRT-PCRs for transgenic Flag-human YAP in the indicated samples. Data are normalized to Gapdh expression and are presented as mean+s.d. of two independent replicates;

FIG. 7b shows representative images of MaSCs or yMaSCs organoids (derived from YAPwt, YAP5SA or TAZ4SA, as indicated) 14 days after transfer to 100% Matrigel/organoid medium (see Methods). Since then, organoids were grown, maintained and passaged without doxycycline. Scale bar, 250 μm;

FIG. 7c shows organoids from MaSCs (positive control) and the indicated yMaSCs expressed E-cadherin by confocal immunofluorescence on frozen sections. Scale bar, 18 μm;

FIG. 7d-e, show a compendium of FIG. 2b.

FIG. 7d shows a schematic representation of the genetic lineage tracing strategy to trace LD cells ex-vivo.

FIG. 7e shows immunostainings of YFP in basal cells (K14-positive) and luminal cells (K8-positive) in yMaSC-derived organoids obtained as in FIG. 2b of the Main Text. Scale bar, 49 μm;

FIG. 7f-h show organoids from the indicated yMaSCs expressed basal/stem (K14, α-SMA, p63) and luminal (K8, K19) markers by confocal immunofluorescence on frozen sections. Scale bars, 17 μm;

FIG. 7i shows a Compendium of FIG. 2f. Treatment with prolactin triggers α-casein expression in MaSC-(control) and yMaSC-derived organoids, as monitored by qRT-PCR. Data are normalized to Gapdh expression. Untreated samples were set to 1. Results are representative of two independent experiments, each performed in triplicate. Data are mean+s.d.; and

FIG. 7j shows a basal population from organoids derived from yMaSCs was sorted with the same markers used to sort the fresh mammary gland and compared by qRT-PCR with freshly sorted LD cells or MaSCs. Data are normalized to Gapdh expression and are referred to MaSC levels for basal genes and to LD levels for all the luminal markers (each set to 1).

FIG. 8 shows the characterization of mammary gland outgrowths derived from MaSCs and yMaSCS; wherein

FIG. 8a shows how yMaSCs were obtained from Yap^{fl/fl}; Taz^{fl/fl} cells. Cells were allowed to form organoids and, during passaging at the single cell level, transduced with Ad-Cre or Ad-GFP as control. Panels are representative images of resulting outgrowths;

FIG. 8b shows Panels which are western blots for YAP and TAZ of lysates from the indicated cells. Lane 1: FACS-sorted LD cells. Lane 2: yMaSCs (wtYAP) after seven days of doxycycline treatment (as in FIG. 1d); tagged Flag-hYAP (with a higher MW than endogenous YAP) is induced. Lane 3: organoids from yMaSCs cultured in the absence of doxycycline (Flag-hYAP turned off, but endogenous YAP/TAZ are expressed). Lane 4: control of endogenous MaSCs. GAPDH serves as loading control;

FIG. 8c refers to FIG. 2h. Representative images of whole-mount hematoxylin staining of cleared fat pad with reconstituted mammary trees from transplanted yMaSCs (from wtYAP), native MaSCs (positive control) and rtTA/EGFP control LD cells (negative control). Scale bar, 0.5 cm; and

FIG. 8d refers to FIG. 2j. Representative sections of virgin mammary gland tree derived from injected MaSCs stained for GFP, K14 and K8. Scale bar, 21 μm.

FIG. 9 shows properties of in vitro-propagated NSCs and yNSC; wherein

FIG. 9a-b, refer to FIG. 3a,b. Representative confocal images of endogenous TAZ costained with Nestin in primary NSCs (a) or with TuJ1 in primary neurons (b). Nuclei were stained with DAPI. Scale bar, 23 μm;

FIG. 9c shows qRT-PCRs for the known YAP/TAZ targets genes Axl, Cyr61 and AmotL2 in neurons and NSCs (mean+s.d). Results are representative of three independent experiments performed in triplicate. Data were normalized to Gapdh expression;

FIG. 9d show representative images of neurospheres from wild-type (wt) or Yap^{fl/fl}; Taz^{fl/fl} NSCs transduced with Ad-Cre. Scale bar, 250 μm;

FIG. 9e shows a schematic representation of the Cre-excisable constructs that express constitutive rtTA or doxy-inducible Flag-human wild-type YAP. Upon integration in the cellular genome, the whole viral cassette gets flanked by LoxP sites; this enables its subsequent Cre-mediated excision;

FIG. 9f-h show how Neurons were transduced with the above Cre-exisable vectors encoding for rtTA and doxycycline-inducible YAP wt, and treated to obtain P0 yNSCs. P0 yNSCs were dissociated at the single cell level and replated in NSC medium+doxycycline to allow P1 yNSCs formation with or without Ad-Cre. f, the panel includes representative images of the yNSCs, before and post-excision. Scale bar, 210 μm. g, Flag-human YAP could not be detected post-excision. GAPDH serves as loading control. h, quantification of neurospheres from yNSCs post-excision in two serial passages. Results are representative of two independent experiments, each performed in six replicates. Data are mean+s.d.;

FIG. 9i shows Panels which are western blots for YAP and TAZ from protein extracts of the indicated cells. Lane 1: neurons. Lane 2: yNSCs (P0) were obtained using excisable YAP transgene, and maintained in Doxy; as in Extended Data FIG. 5f. Cells (from P2-to-P3) were plated as monolayer in presence of Doxy and lysed after 1 day. Lane 3: the same yNSCs of lane 2, kept in absence of Doxy from P2. Lane 4: yNSCs as in lane 2, but after excision of the viral cassette (at P1, as in Extended Data FIG. 5f). Lane 5: lysates of NSCs as comparison; and

FIG. 9j shows how yNSCs (passage 4 as neurospheres) were dissociated, plated on fibronectin-coated dishes and transfected with the indicated siRNAs. The panel represents the quantification of neurospheres derived from the indicated cells.

FIG. 10 shows expression of YAP converts neurons in NSC-like cells; wherein

FIG. 10a refers to FIG. 3h of the main text and to FIG. 10 b-d. Schematic representation of the genetic lineage tracing strategy used to trace neurons ex-vivo;

FIG. 10b-d shows a lineage tracing experiment with the Thy1-Cre driver showing that yNSCs originate from neurons. b, immunostaining for GFP and TuJ1 in neurons obtained from Thy1-Cre; R26-LSL-rtTA-IRES-EGFP hippocampi. c, bright field and GFP-fluorescence pictures of yNSCs obtained from neurons in b after transduction with doxycycline-inducible YAP wt. d, immunostainings of yNSCs as in c showing positivity for GFP and neural stem cell markers Nestin, SOX2 and Vimentin. Scale bars in b,d, 37 μm, in c, 105 μm;

FIG. 10e shows a lineage tracing experiment with the Syn1-Cre driver showing that yNSCs originate from neurons. b, immunostaining for GFP and TuJ1 in neurons obtained from Syn1-Cre; R26-LSL-rtTA-IRES-EGFP corteces. c, bright field and GFP-fluorescence pictures of yNSCs obtained from neurons in e after transduction with doxycycline-inducible YAP wt.

FIG. 11 shows Differentiation of yNSCs; wherein

FIG. 11a refers to FIG. 3l. yNSCs were plated and differentiated toward a neuronal fate (see Methods). Similar experiments carried out with endogenous, tissue-derived NSCs are presented as reference. Panels represent confocal images for neuronal differentiation markerTau. Scale bars, 50 μm;

FIG. 11 b-c refers to FIG. 3l and to FIG. 11a. yNSCs differentiated toward a neuronal fate (b, TuJ1-positive; c, Tau-positive) were negative for Nestin, as showed by immunofluorescence. Similar results were obtained with endogenous NSCs (data not shown). Scale bars, 9 μm;

FIG. 11d-g shows yNSCs which were transduced with a constitutive EGFP-expressing vector and injected in the brains of recipient mice. Four weeks later, brains were fixed and processed for immunofluorescence analyses. d, Panels are representative confocal images showing that injected cells (GFP-positive) lost expression of the NSC marker Nestin. A field of the subventricular zone (SVZ) of the same brain sections is shown as positive control of the Nestin staining. e-g, Representative confocal images of yNSCs injected in the brain of recipient mice, showing injected cells (GFP-positive) stained for GFAP (e), NeuN or Tuj1 (f) and CNPase (g). Scale bars, 19 μm.

FIG. 12 shows how YAP expression converts pancreatic acinar explants to duct-like organoids. This refers to FIG. 4a-d:

FIG. 12a show Pancreatic ductal organoids (Ducts, bottom panels) which display nuclear YAP/TAZ by immunofluorescence. Primary pancreatic acini (top panels) are presented as reference. Scale bar, 80 μm;

FIG. 12b shows qRT-PCRs for the known YAP/TAZ targets genes Axl, Ctgf and AnkrD1 in primary pancreatic acini and pancreatic ductal orgnaoids (Ducts) (mean+s.d). Results are representative of three independent experiments performed in triplicate. Data were normalized to 18-S rRNA expression;

FIG. 12c, shows Ducts which were derived from wild-type (wt) or Yap; Taz^{fl/fl} mice and, during passaging at the single cell level, transduced with Ad-Cre or Ad-GFP as control. Panels are representative images of resulting outgrowths. Scale bars, 70 μm;

FIG. 12d shows a schematic representation of the experiments performed with pancreatic acinar explants. Pancreatic acini were isolated from R26-rtTA; tetO-YAP^{S127A} mice and either plated as single cells in Matrigel or seeded as whole acini in 3D collagen (see Methods). Acinar cells were cultured in the presence of doxycycline (DOXY) until primary organoids appeared. Organoids obtained from both culture conditions were then passaged in fresh Matrigel in the absence of doxycycline (WITHOUT DOXY) every 10 days;

FIG. 12e refers to FIG. 4c. Quantification of primary organoids arising from R26-rtTA; tetO-YAP^{S127A} single acinar cells treated as indicated in d. Negative controls—acinar cells derived from R26-rtTA; tetO-YAP^{S127A} mice and cultured in absence of doxycycline or acinar cells derived from R26-rtTA mice—never formed organoids. Data are presented as mean+s.d. and are representative of five independent experiments, performed with four technical replicates;

FIG. 12 f-g show serial images of a whole acinus derived from R26-rtTA; tetO-YAP^{S127A} growing as cyst-like organoid at the indicated time points after Doxycycline addition (f) and after one or four passages in fresh Matrigel in the absence of Doxycycline (g). Scale bars, 70 μm in f; 290 μm in g; and

FIG. 12h shows quantification of the ability of whole acini to form ductal organoids upon transgenic YAP overexpression as in Extended Data FIG. 8f. Data are presented as mean+s.d. and are representative of five independent experiments, performed with four technical replicates.

FIG. 13 shows lineage tracing of pancreatic acinar explants conversion to duct-like organoids upon YAP expression; this refers to FIG. 4e-h of the main text; wherein

FIG. 13a shows a schematic representation of the experiments performed with pancreatic acinar explants for lineage tracing. Pancreatic acini were isolated from Ptf1a-CreERTM; R26-LSL-rtTA-IRES-EGFP; tetO-YAP^{S127A} mice and either plated as single cells in Matrigel or seeded as whole acini in 3D collagen (see Methods). Acinar cells were cultured in the presence of doxycycline (DOXY) until primary organoids appeared. Organoids obtained from both culture conditions were then passaged in fresh Matrigel in the absence of doxycycline (WITHOUT DOXY) every 10 days. Negative controls—acinar cells derived from Ptf1a-CreERTM; R26-LSL-rtTA-IRES-EGFP; tetO-YAP^{S127A} mice and cultured in absence of doxycycline—never formed organoids (b,c);

FIG. 13b refers to FIG. 4e. Panels are bright field and GFP-fluorescence pictures of transgenic Ptf1a-CreERTM; R26-LSL-rtTA-IRES-EGFP; tetO-YAP^{S127A} exocrine cells, at the indicated time points in absence of Doxy treatment (Negative control). Scale bar, 33 μm;

FIG. 13c shows Panels which are bright field and GFP-fluorescence pictures of Ptf1a-CreERTM; R26-LSL-rtTA-IRES-EGFP; tetO-YAP^{S127A} whole exocrine acini, at the indicated time points in absence of Doxy treatment (Negative controls). Scale bar, 33 μm;

FIG. 13d-e show lineage-tracing experiments using the Ptf1a-CreERTM driver. Panels are bright field and GFP-fluorescence pictures of transgenic YAP-expressing whole exocrine acini derived from Ptf1a-CreERTM; R26-LSL-rtTA-IRES-EGFP; tetO-YAP^{S127A} mice, at the indicated time points of Doxy treatment (d) and after passaging in absence of Doxy in fresh Matrigel (e). The same acini formed no organoidsin absence of doxycycline (c). Scale bars, 33 μm in d; 70 μm in e;

FIG. 13 f shows qRT-PCRs for the indicated exocrine and Ductal/progenitor markers in fresh pancreatic acini, yDucts and Ducts. Data are normalized to 18SrRNA expression and are referred to Acini for exocrine differentiation markers, and to Ducts for Ductal/progenitor genes (each set to 1). Results are representative of four independent experiments performed in triplicate. Data are presented as mean+s.d.; and

FIG. 13 g shows a representative immunofluorescences for the ductal marker K19 and the exocrine marker CPA1 before (day 0) and after yDuct differentiation (day 8). Similar results were obtained with organoids from normal ducts (not shown). Scale bar: 17 μm.

FIG. 14 shows that ySCs do not express pluripotency markers; wherein

FIG. 14a shows qRT-PCRs for the pluripotency factors Oct4, Nanog and Sox2 in the indicated samples. Mouse embryonic stem cells are used as reference. Results are representative of two independent experiments performed in triplicate. Data are presented as mean+s.d.

### DETAILED DESCRIPTION OF EXEMPLARY EMBODIMENTS

In one aspect, the present invention provides a method for generating somatic stem cells, comprising the steps of:
a. providing at least one differentiated cell, committed progenitor or partially differentiated cell;
b. inducing an increased expression or activity of a YAP protein, and/or a TAZ protein, and/or a functional fragment of the YAP and/or the TAZ protein, and/or an activated version of the YAP and/or the TAZ protein, or derivatives thereof in at least one differentiated cell or committed progenitor or partially differentiated cell;
c. generating a somatic stem cell out of said differentiated cell, committed progenitor or partially differentiated cell.

While induction of one of the increased expression or activity of a YAP protein, or a TAZ protein, or a functional fragment of the YAP or the TAZ protein, or an activated version of the YAP or the TAZ protein, or derivatives thereof, is sufficient to generate a somatic stem cell out of said differentiated cell, committed progenitor or partially differentiated cell, a combination of induced increase of expression of multiple of the proteins is also contemplated.

In some embodiments, the method comprises inducing the increased expression of the YAP protein, and/or the TAZ protein, and/or the functional fragment of the YAP and/or the TAZ protein, and/or the activated version of the YAP and/or the TAZ protein, or derivatives thereof in at least one differentiated cell or committed progenitor or partially differentiated cell (starting cell) transiently.

In some embodiments, the expression of the YAP protein, and/or the TAZ protein, and/or the functional fragment of the YAP and/or the TAZ protein, and/or the activated version of the YAP and/or the TAZ protein, or derivatives thereof in said at least one differentiated cell or committed progenitor or partially differentiated cell is increased transiently for a time sufficient for inducing the generation of a somatic stem cell out of the starting cell.

Once the induction of the generation of the somatic stem cell out of the starting cell has been initiated by the transient increase of expression of the YAP protein, and/or the TAZ protein, and/or the functional fragment of the YAP and/or the TAZ protein, and/or the activated version of the YAP and/or the TAZ protein, or derivatives thereof, the induced transient increase may be reduced and/or terminated.

Whilst not being bound by theory, it is thought that induced transient increase of expression of the YAP protein, and/or the TAZ protein, and/or the functional fragment of the YAP and/or the TAZ protein, and/or the activated version of the YAP and/or the TAZ protein, or derivatives thereof amongst other functions leading to the generation of a somatic stem cell out of the starting cell, initiates an expression of endogenous YAP/TAZ which is sufficient for maintaining stem cell properties in the generated somatic stem cell.

Transient induction of expression may thus be advantageously used in the method according to the present invention in order to improve security of the method. For example, obtained somatic stem cells may be used with reduced risk for adverse effects in regenerative medicine applications.

According to one embodiment, increased expression or increased activity of at least one endogenous YAP/TAZ protein in the cell is induced in the starting cell. This may be done by applying to the at least one differentiated cell, or committed progenitor, or partially differentiated cell a composition comprising a substance for influencing the biological activity of the endogenous YAP/TAZ protein, and/or for influencing a cellular stability of the endogenous YAP/TAZ protein, and/or for influencing a cellular localization of the endogenous YAP/TAZ protein. For example, inhibitors of endogenous expression of YAP/TAZ proteins in the starting cell may be blocked by the substance, or activation pathways for increase of expression of the endogenous YAP/TAZ proteins in the at least one differentiated cell may be activated by the substance.

The substance may activate a biological activity of the endogenous YAP/TAZ protein, by modulating e.g. a conformation and/or a modification of the endogenous YAP/TAZ protein. The substance may modulate the cellular localization of endogenous YAP/TAZ protein or increase the stability of endogenous YAP/TAZ protein. For example, the endogenous YAP/TAZ protein may be protected from degradation/digestion from cellular proteins. For example, the biological activity of the endogenous YAP/TAZ protein being influenced by the substance may be transcriptional activity of the endogenous YAP/TAZ protein. It is also contemplated that the substance modulates a histone modification for inducing increased expression of the endogenous YAP/TAZ protein. Of course, other generally known ways to induce an increase in gene expression may also be used by the substance for influencing the biological activity of the endogenous YAP/TAZ protein.

In a preferred embodiment, the increased expression and/or increased activity of the at least one endogenous YAP/TAZ protein in the cell is induced transiently.

According to one embodiment, in combination with or alternative to applying a substance to the starting cell in order to activate a biological activity of endogenous YAP/TAZ protein, the starting cell may be transfected with a vector comprising a nucleotide sequence coding for a protein which induces an increased expression or a biological activity of said endogenous YAP/TAZ protein.

The biological activity of endogenous YAP/TAZ is understood to be a biological activity which leads to the generation of somatic stem cells out of the at least one differentiated cell or committed progenitor or partially differentiated cell in accordance with the method of the present invention.

In one embodiment, the increased expression of said YAP/TAZ protein and/or said functional fragment and/or said activated version in said at least one differentiated cell is ectopic.

In a preferred embodiment, said at least one differentiated cell of step a) is transfected with a vector comprising a nucleotide sequence coding for a wild-type YAP protein and/or a nucleotide sequence coding for a wild-type TAZ protein and/or a nucleotide sequence coding for a functional fragment of said wild-type YAP protein and/or said wild-type TAZ protein, and/or a nucleotide sequence coding for said activated version, or derivatives thereof.

Preferably, a nucleotide sequence coding for a wild-type YAP protein is used as set forth in Seq. ID No 1.

Preferably, a nucleotide sequence coding for a wild-type TAZ protein is used as set forth in Seq. ID No 2.

Preferably, a nucleotide sequence coding for an activated version of the YAP protein is used as set forth in Seq. ID No 3.

Preferably, a nucleotide sequence coding for an activated version of the TAZ protein is used as set forth in Seq. ID No 4.

In yet other specific embodiments, the present invention provides a vector comprising a nucleotide sequence having at least 70%, 80%, 90%, or 95% identity to at least 60 nucleotides of the sequences set forth in SEQ ID No's 1, 2, 3 or 4.

The transfection of said at least one differentiated cell may be performed using a lentiviral vector. Further, the expression of said wild-type YAP/TAZ protein and/or said functional fragment and or/said activated version may be under the control of an inducible promoter. For example, said inducible promoter may be a doxycyclin-inducible promoter. Such promoter has been described e.g. in U.S. Pat. Nos. 5,814,618, 7,541,446, and 8,383,364. However, other inducible promoter-systems which are generally known in the art are also contemplated. Use of this vector system allows for easy controlling of the transient induction period for increased expression of said wild-type YAP/TAZ protein and/or said functional fragment and or/said activated version.

In a preferred embodiment of the present application, a doxycyclin inducible promoter is used according to a nucleotide sequence as set forth in Seq ID No 5. Such tetO promoter system has been described e.g. by Bujard, Hermann and M. Gossen (“Tight Control of Gene Expression in Mammalian Cells by Tetracycline-Responsive Promoters; (Proc. Natl. Acad. Sci. U.S.A. 89 (12): 5547-51).

If the somatic stem cell has been generated by carrying out step b) of the method according to the present invention for a sufficient time, the transfected nucleotide sequence coding for the wild-type YAP protein and/or the nucleotide sequence coding for the wild-type TAZ protein and/or the nucleotide sequence coding for the functional fragment of said wild-type YAP protein and/or said wild-type TAZ protein, and/or the nucleotide sequence coding for said activated version or derivatives thereof may be removed from the generated somatic stem cell. Such removal of the transfected nucleotide sequence may be carried out according to the standard methods known in the art, depending on the vector system used for transfection.

As used herein, the term “sufficient time” shall mean a period sufficiently long to reprogram the differentiated cell by the transient induction of increased expression of the YAP/TAZ proteins disclosed herein.

In some embodiments, the term “sufficient time” shall mean at least one day, at least 2 days, at least 3 days, at least 4 days, at least 5 days, at least 6 days, at least 7 days, at least 8 days, at least 9 days, at least 10 days, at least 15 days, or at least 30 days.

In some embodiments, the term “sufficient time” ranges from 1 day to about 180 days, e.g., from about 1 day to about 2 days, from about 1 day to about 7 days, from about 1 day to about 14 days, from about 1 day to about 21 days, from about 1 day to about 30 days, from about 1 day to about 45 days, from about 1 day to about 60 days, from about 1 day to about 90 days, from about 1 day to about 120 days, from about 1 day to about 150 days, or from about 1 day to about 180 days.

In some embodiments, the term “sufficient time” ranges from 2 days to about 180 days, e.g., from about 2 days to about 7 days, from about 2 days to about 14 days, from about 2 days to about 21 days, from about 2 days to about 30 days, from about 2 days to about 45 days, from about 2 days to about 60 days, from about 2 days to about 90 days, from about 2 days to about 120 days, from about 2 days to about 150 days, or from about 2 days to about 180 days.

As further used herein, the term vector is understood to mean any DNA molecule that can be used as a vehicle to artificially carry foreign genetic material into another cell, where it can be replicated and/or expressed.

As further used herein, the term functional fragment is understood to mean a truncated and/or incomplete form of a YAP/TAZ protein which still harbors its functional activity to induce de novo generation of a somatic stem cell out of a more differentiated cell.

In another aspect of the present invention, it is provided a somatic stem cell obtained by the method according to the present invention. The induced somatic stem cell according to the present invention may be used in a regenerative medicine application. For example, the somatic stem cells may be used for generating tissues for transplantation. The somatic stem cells may be used to repair or replace tissue or organ function lost due to age, disease, organ damage, or congenital defects. The induced somatic stem cells may then be used to generate cells and tissue ex-vivo, to correct genetic defects, to expand or generate de novo stem cells in vivo, including self-propagating cells with augmented properties in comparison with natural/endogenous stem cells.

In another aspect of the present invention, a vector for use in the method of the present application is provided; the vector comprising a nucleotide sequence coding for a wild-type YAP protein, and/or a nucleotide sequence coding for a wild-type TAZ protein, and/or a nucleotide sequence coding for a functional fragment of said YAP and/or said TAZ protein, and/or a nucleotide sequence coding for an activated version of said YAP and/or said TAZ protein, wherein the transcription of said nucleotide sequence is under the control of an inducible promoter.

In accordance with the present invention, the inhibitor (i.e. in case of a nucleic acid inhibitor) of the polynucleotide to be inhibited in context of the present invention may be cloned into a vector. The term “vector” as used herein particularly refers to plasmids, cosmids, viruses, bacteriophages and other vectors commonly used in genetic engineering. In a preferred embodiment, these vectors are suitable for the transformation of cells, like fungal cells, cells of microorganisms such as yeast or prokaryotic cells. In a particularly preferred embodiment, such vectors are suitable for stable transformation of bacterial cells, for example to transcribe the polynucleotide of the present invention.

Accordingly, in one aspect of the invention, the vector as provided is an expression vector. Generally, expression vectors have been widely described in the literature. As a rule, they may not only contain a selection marker gene and a replication-origin ensuring replication in the host selected, but also a promoter, and in most cases a termination signal for transcription. Between the promoter and the termination signal there is preferably at least one restriction site or a polylinker which enables the insertion of a nucleic acid sequence/molecule desired to be expressed.

It is to be understood that when the vector provided herein is generated by taking advantage of an expression vector known in the prior art that already comprises a promoter suitable to be employed in context of this invention, for example expression of an inhibitor (i.e. in case of a nucleic acid inhibitor) of a polynucleotide as described hereinabove, the nucleic acid construct is inserted into that vector in a manner the resulting vector comprises only one promoter suitable to be employed in context of this invention. The skilled person knows how such insertion can be put into practice. For example, the promoter can be excised either from the nucleic acid construct or from the expression vector prior to ligation.

As a non-limiting example, a vector comprising a nucleotide sequence coding for a wild-type YAP protein, and/or a nucleotide sequence coding for a wild-type TAZ protein, and/or a nucleotide sequence coding for a functional fragment of said YAP and/or said TAZ protein, and/or a nucleotide sequence coding for an activated version of said YAP and/or said TAZ protein, and/or a nucleotide sequence coding for a protein which induces an increased expression or activity of an endogenous YAP/TAZ protein, or derivatives thereof, is cloned is an adenoviral, adeno-associated viral (AAV), retroviral, or nonviral minicircle-vector. Further examples of vectors suitable to comprise an inhibitor (i.e. in case of a nucleic acid inhibitor) of a polynucleotide to be inhibited in order to induce increased expression of an endogenous YAP/TAZ protein in context of the present invention to form the vector described herein are known in the art.

In an additional embodiment, the coding nucleic acid sequence of an inducer of YAP/TAZ in context of the present invention and/or the vector into which the polynucleotide described herein is cloned may be transduced, transformed or transfected or otherwise introduced into a host cell. For example, the host cell is a eukaryotic or a prokaryotic cell, for example, a bacterial cell. As a non-limiting example, the host cell is preferably a mammalian cell. The host cell described herein is intended to be particularly useful for generating the inhibitor of a polynucleotide to be inhibited in context of the present invention. An inducer of YAP/TAZ is intended as a polynucleotide sequence able to activate YAP/TAZ nuclear localization and transcriptional activation (as determined by luciferase assays and activation or YAP/TAZ direct target genes such as CTGF) Dupont et al., Nature 2011).

An overview of examples of different corresponding expression systems to be used for generating the host cell described herein is for instance contained in Methods in Enzymology 153 (1987), 385-516, in Bitter (Methods in Enzymology 153 (1987), 516-544), in Sawers (Applied Microbiology and Biotechnology 46 (1996), 1-9), Billman-Jacobe (Current Opinion in Biotechnology 7 (1996), 500-4), Hockney (Trends in Biotechnology 12 (1994), 456-463), and in Griffiths (Methods in Molecular Biology 75 (1997), 427-440). The transformation or genetically engineering of the host cell with a polynucleotide to be inhibited in context of the present invention or vector described herein can be carried out by standard methods, as for instance described in Sambrook and Russell (2001), Molecular Cloning: A Laboratory Manual, CSH Press, Cold Spring Harbor, N.Y., USA; Methods in Yeast Genetics, A Laboratory Course Manual, Cold Spring Harbor Laboratory Press, 1990.

### EXAMPLES

The following examples illustrate, rather than limit, embodiments of the present invention.

### Example 1: YAP/TAZ Revert Differentiated Cells of the Mammary Gland into MaSC-Like Cells

The mammary gland represents a classic model system for the study of epithelial SCs and tissue regeneration. Remarkably, implantation of mammary gland SCs (MaSCs) into the mammary fat pad is sufficient to regenerate an entire ductal tree, with MaSCs contributing to both the luminal and myoepithelial lineages.

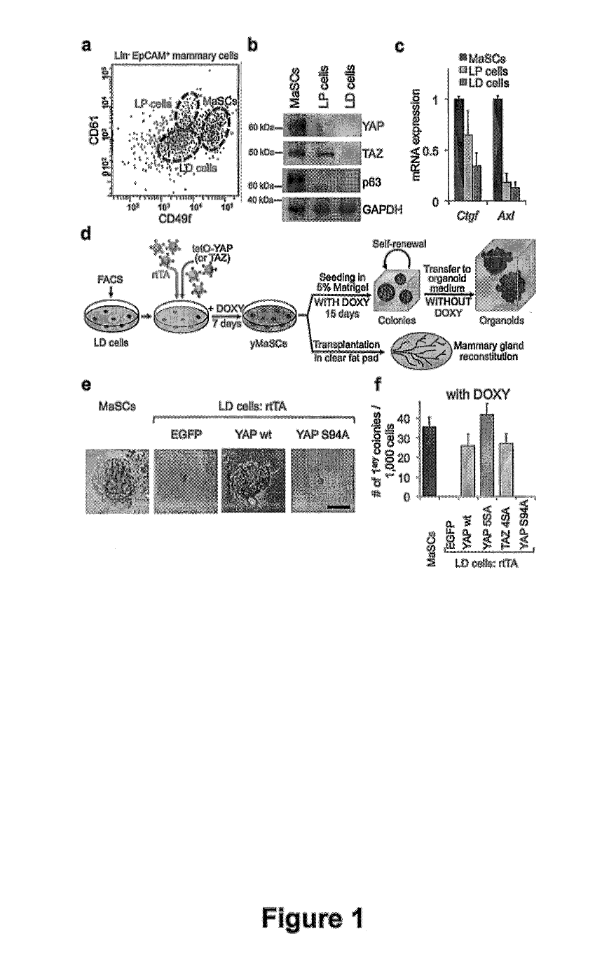

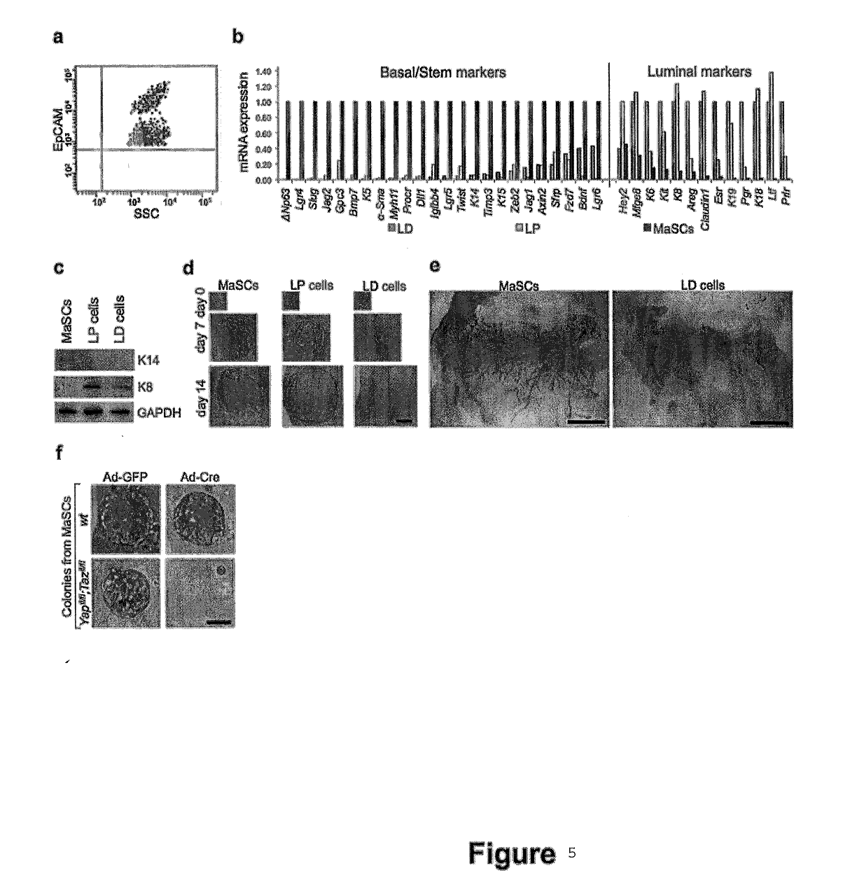

To address whether expression of YAP/TAZ may bestow stemness characteristics also to normal mammary cells, freshly dissected, lineage negative (Lin-) and EpCAM positive mammary epithelial cells were FACS-purified using the CD61 and CD49f cell surface antigen markers (FIG. 1a). As previously described (Guo, W. et al. Slug and Sox9 cooperatively determine the mammary stem cell state; Cell 148, 1015-1028 (2012)) this procedure allowed to distinguish three subpopulations of mammary cells: a MaSC-enriched fraction (EpCAMlowCD49fhighCD61+), luminal progenitors (LP, EpCAMhighCD49flowCD61+), and luminal differentiated cells (LD, EpCAMhighCD49flowCD61−). As expected, the MaSC fraction expressed basal and SC markers, and was the only one able to regenerate a complete mammary ductal tree after transplantation in vivo, whereas LD cells were unable to proliferate (FIG. 5b-e). It has been found that endogenous YAP/TAZ proteins—and their transcriptional targets Ctgf and Ax1—were detected in the MaSC-containing population, but at much lower levels in differentiated cells (FIG. 1b, c). Importantly, YAP/TAZ are the endogenous factors required to sustain the expansion of primary MaSCs in vitro:MaSCs purified from Yapfl/fl; Tazfl/fl mice failed to form any outgrowths and remaining as single cells after genetic ablation of YAP/TAZ ex-vivo by adenoviral delivery of the Cre recombinase (FIG. 5f).

To investigate whether ectopic expression of YAP or TAZ in LD cells could impart MaSC-like properties, FACS-purified LD cells were plated on collagen-coated dishes and transduced with doxycycline-inducible lentiviral vectors encoding for wild-type (wt) YAP, or the activated versions of YAP and TAZ (i.e., YAP5SA or TAZ4SA, lacking inhibitory phosphorylation sites) (see diagram in FIG. 1d). As control, cells were infected with an inducible EGFP vector. Transduced cells were cultured for 7 days in doxycycline-containing medium (see Methods) and then plated at clonogenic density in three-dimensional 5% Matrigel cultures (Shackleton, M. et al. Generation of a functional mammary gland from a single stem cell. Nature 439, 84-88 (2006). EGFP-expressing control cells invariably remained as single cells, without ever originating even a single colony in more than 20 independent experiments (FIG. 1e-f). Strikingly, cells expressing either YAP or TAZ formed solid outgrowths similar to those generated by MaSCs (FIG. 1e, f). As further control, expression of transcriptionally deficient YAPS94A had no effect.

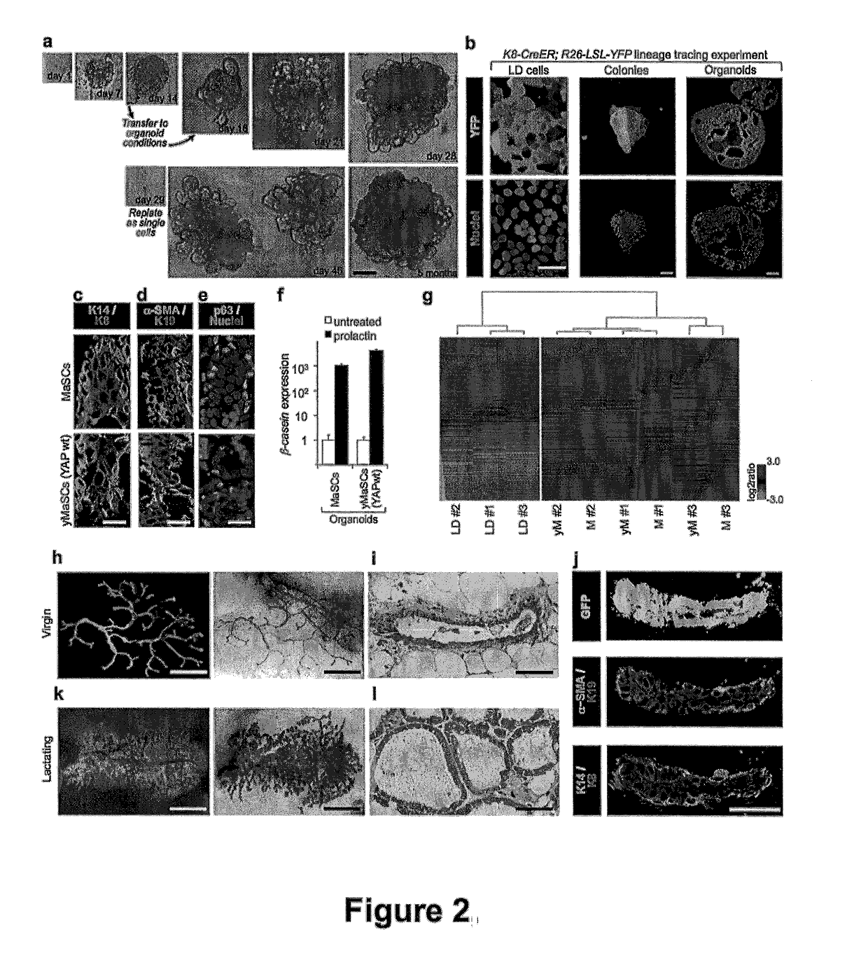

It has been examined whether increased YAP/TAZ expression may convert luminal cells to a MaSC-like state. First, it has been addressed whether YAP/TAZ expression endowed self-renewal potential, a fundamental SC trait that can be assayed in vitro by the ability to serially passage mammary colonies. YAP/TAZ-induced colonies, similarly to those generated from MaSCs, could form additional generations of colonies after single cell dissociation. Notably, the colony-forming efficiency after passaging was comparable in presence and absence of doxycycline, that is, irrespective of ectopic YAP/TAZ expression (Extended Data FIG. 2a,b). This suggests that transient expression of YAP/TAZ is sufficient to stably endow self-renewal potential to mammary epithelial cells.

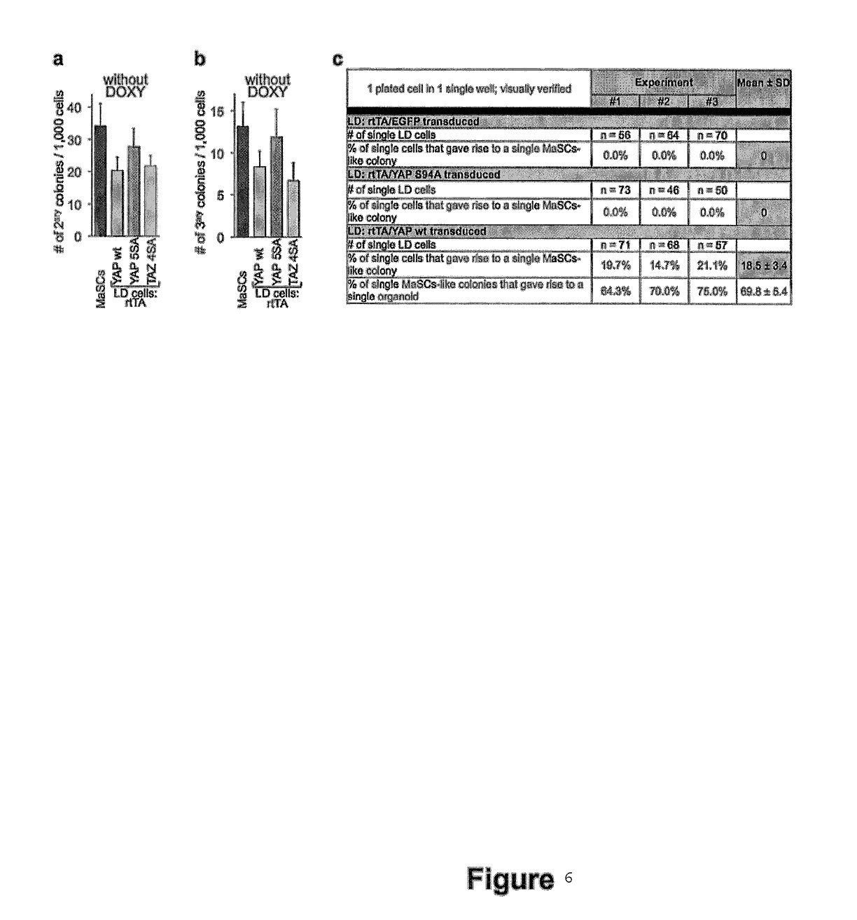

To verify whether the switch from LD to a MaSC-like state could be recapitulated at the single cell level, individual LD cells were seeded in 96-well plates (visually verified) and induced to express YAP. By monitoring the resulting outgrowths, it has been found that 18% of these individual cells formed solid colonies (FIG. 6c) that could be further passaged as clonal organoids (FIG. 2a and see below). Hereafter, YAP/TAZ induced “MaSC-like” cells are thus designated as “yMaSCs”.

### Example 2: The Expansion, Differentiation and Regenerative Potential of yMaSCs

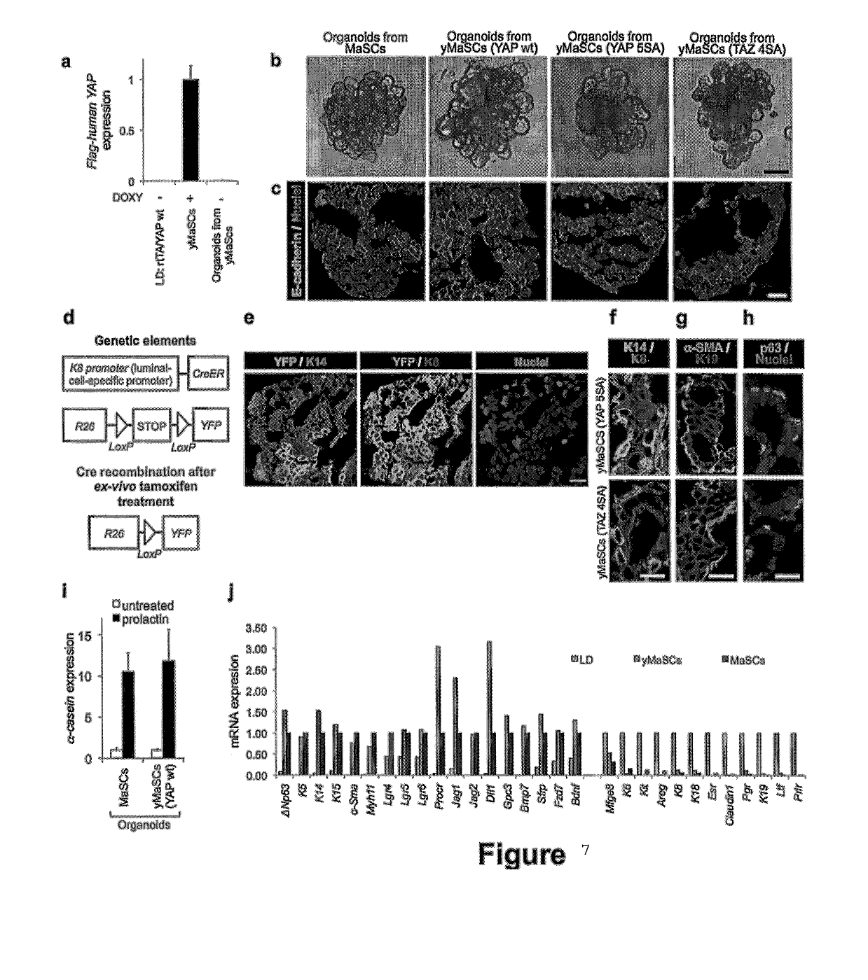

It was then established if yMaSCs truly represented mammary SCs, as determined by additional cardinal properties of SCs, such as the ability to self-organize in vitro into mammary tissue-like structures, to differentiate along distinct lineages, and to regenerate a mammary tree in vivo after injection into a cleared mammary fat pad. For this, a long-term culture system has been established that allows yMaSCs to form mammary-gland like structures in vitro. MaSC- and yMaSC-derived colonies were transferred and embedded into 100% Matrigel, and overlaid with “organoid” medium containing EGF, bFGF, Noggin, B27, and R-Spondin112 in absence of doxycycline. Under these conditions, colonies underwent extensive budding and, by 2 weeks, grew into large epithelial organoids (FIG. 2a and FIG. 7b-c). Organoids derived from yMaSC colonies developed at high frequency (about 70%), and were indistinguishable in growth pattern and size to those generated by natural MaSCs.

To further validate the notion that YAP expression converts differentiated cells to a SC fate genetic lineage-tracing experiments have been carried out using LD cells irreversibly labeled with YFP purified from K8-CreERT2; R26-LSL-YFP mice (FIG. 7d) (Van Keymeulen, A. et al. Distinct stem cells contribute to mammary gland development and maintenance. Nature 479, 189-193 (2011)). As shown in FIG. 2b, organoids generated by YAP-reprogramming of these cells were entirely YFP-positive, attesting their origin from the luminal lineage. By histological examination, organoids were composed by a stratified epithelium (E-cadherin positive, FIG. 7c). Internal cells, surrounding a lumen-like cavity, expressed differentiated luminal markers such as K8 and K19 (FIG. 2c-e). Outer cells (either at external surfaces or bordering inner folds) displayed expression of basal, myoepithelial and SC markers (K14, α-smooth muscle actin/α-SMA, p63); notably, as validated by K8-CreERT2; R26-LSL-YFP lineage tracing, basal/K14-positive cells were generated de-novo from YAP-reprogrammed LD cells (FIG. 7d-e). Furthermore, addition of a lactogenic stimulus triggered expression of α- and β-casein, indicative of alveolar (milk-producing) cell differentiation (FIG. 2f and FIG. 7i). yMaSC-derived organoids were dissociated, replated as single cells every 2 weeks and cultured for at least 9 months without changes in growth pattern, plating efficiency and differentiation potentials. Taken together the results indicate that, similarly to authentic MaSCs, yMaSCs i) display long-term self-renewal potential, ii) generate self-organizing epithelial structures reminiscent of the normal mammary gland and iii) retain multilineage differentiation ability.

To characterize at the molecular level the similarities between yMaSCs to their natural counterpart, we compared FACS-purified SCs (EpCAMlowCD49fhighCD61+) from the mammary gland and yMaSC-induced organoids. Purified LD cells were used as control. As shown in FIG. 7j, MaSCs and yMaSCs display a remarkable overlap in gene expression: in yMaSCs, luminal differentiation markers (such as such as Esr, claudin or K18/K19) were downregulated, and basal markers induced (such as K14 and a-SMA), all to levels comparable to those of endogenous MaSCs, suggestive of a complete reprogramming of LD cells to a SC-state. Importantly, this also applies to genes previously associated to various types of mammary SCs, including SC markers·Np63, LGR4/5/6, Procr, and the myoepithelial marker Myh11, recently associated to some mammary repopulating units (Wang, D. et al. Identification of multipotent mammary stem cells by protein C receptor expression. Nature 517, 81-84 (2015)). It may thus be concluded that yMaSCs integrate the molecular traits variously attributed to MaSCs in prior publications.

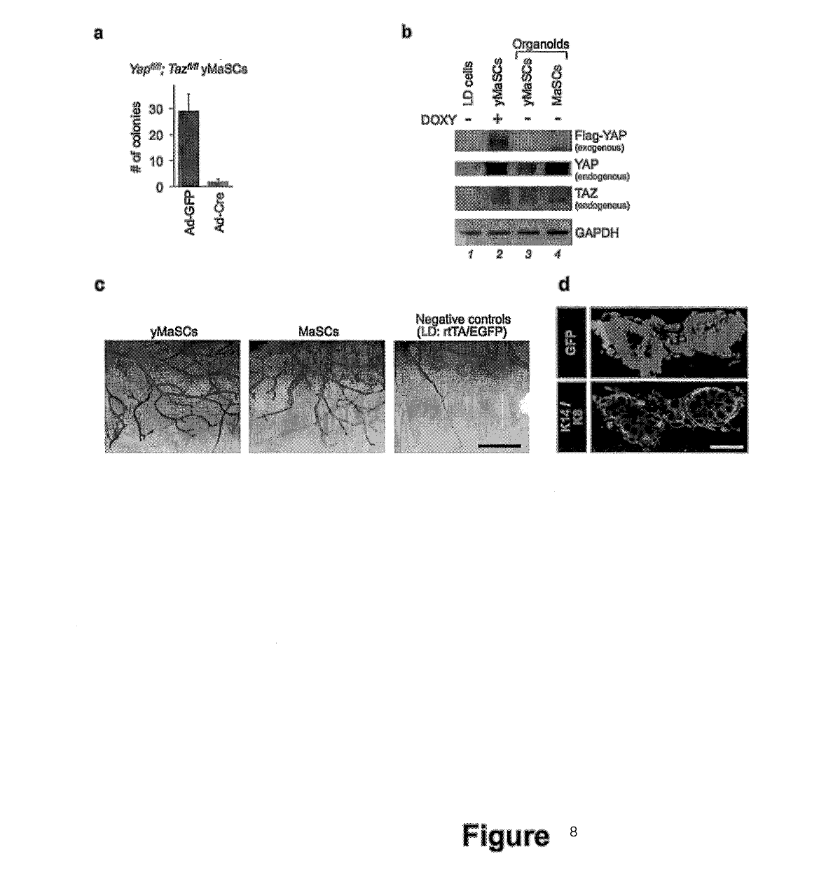

To reinforce the notion that MaSCs and yMaSCs are similar, the gene-expression profiles of their respective organoid cultures, and of LD cells have been compared (FIG. 2g). By unsupervised hierarchical clustering of differentially expressed genes, organoids from MaSCs or yMaSCs could not be distinguished. At the functional level, and similarly to native MaSCs, yMaSCs rely on endogenous YAP/TAZ to preserve their organoid-forming potential, as passaging of organoids from yMaSCs generated from Yapfl/fl; Tazfl/flLD cells is severely affected after Cre-mediated deletion of YAP/TAZ (FIG. 8a). Consistently, it has also been found that conversion to the ySC state was accompanied by activation of endogenous YAP/TAZ proteins. As shown in FIG. 8b, exogenous YAP turned on expression of endogenous YAP and TAZ in LD cells; importantly, these remained expressed in ySC-derived organoids after ectopic YAP expression had been turned off (FIG. 8b). It may thus be concluded that transient exposure to YAP/TAZ is sufficient to empower a self-sustaining loop of endogenous YAP/TAZ expression that recapitulates the natural condition of native MaSCs.

Next, it has been tested whether yMaSCs displayed mammary gland reconstituting activity. For this, FACS-purified LD cells were transduced with vectors encoding for EGFP and inducible wild-type YAP. Cells were treated with doxycycline for 7 days and then transplanted (103-104 cells) into the cleared mammary fat pad of NOD-SCID mice, kept in a doxycycline-free diet for 10 weeks. Strikingly, cells that had experienced transient expression of wild-type YAP had also acquired the ability to regenerate the mammary gland (25%, n=16) (FIG. 2h, i). Ductal tree and terminal end buds were regenerated when as few as 100 YAP-infected LD cells were implanted (33%, n=6). As control, LD cells transduced with the sole EGFP vector did not display any reconstituting activity, at any inoculum dose (0%, n=28, 102-5×104) (FIG. 8c). Histological analyses revealed that the epithelial outgrowths obtained from yMaSCs were EGFP-positive and morphologically indistinguishable from those generated by endogenous MaSCs, and consisted of a bilayered epithelium, composed of a basal/myoepithelial layer (positive for K14 and α-SMA) overlaid by luminal cells (positive for K8) (FIG. 2j).

To explore the reconstituting potential of a single yMaSC, in the cleared fat pads single-cell derived organoids have been injected, and it was found that these were also able to regenerate the mammary gland (33%, n=6). Notably, when these mice were impregnated, reconstituted mammary glands generated a dense ductal system ending in clusters of milk-secreting alveoli, indicating that yMaSCs retain full differentiation potential in vivo (FIG. 2k, l). It may thus be concluded from this collective set of experiments that transient expression of YAP/TAZ in differentiated cells of the mammary gland is sufficient to convert them into bona fide MaSCs.

### Example 3: YAP Turns Neurons into Neural SC-Like Cells

Next, it was checked whether SC-generation by ectopic YAP expression was specific for mammary epithelial cells, or rather represented a more general principle. This question has been addressed in neurons, a cell type considered a classic example of terminal differentiation.

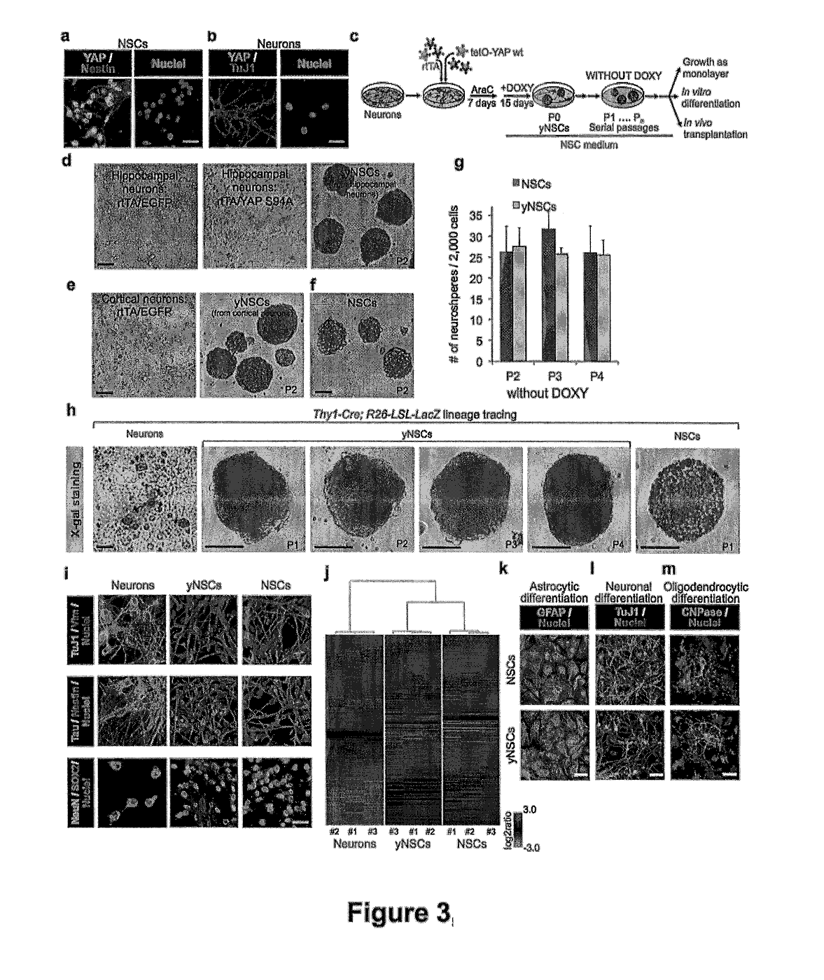

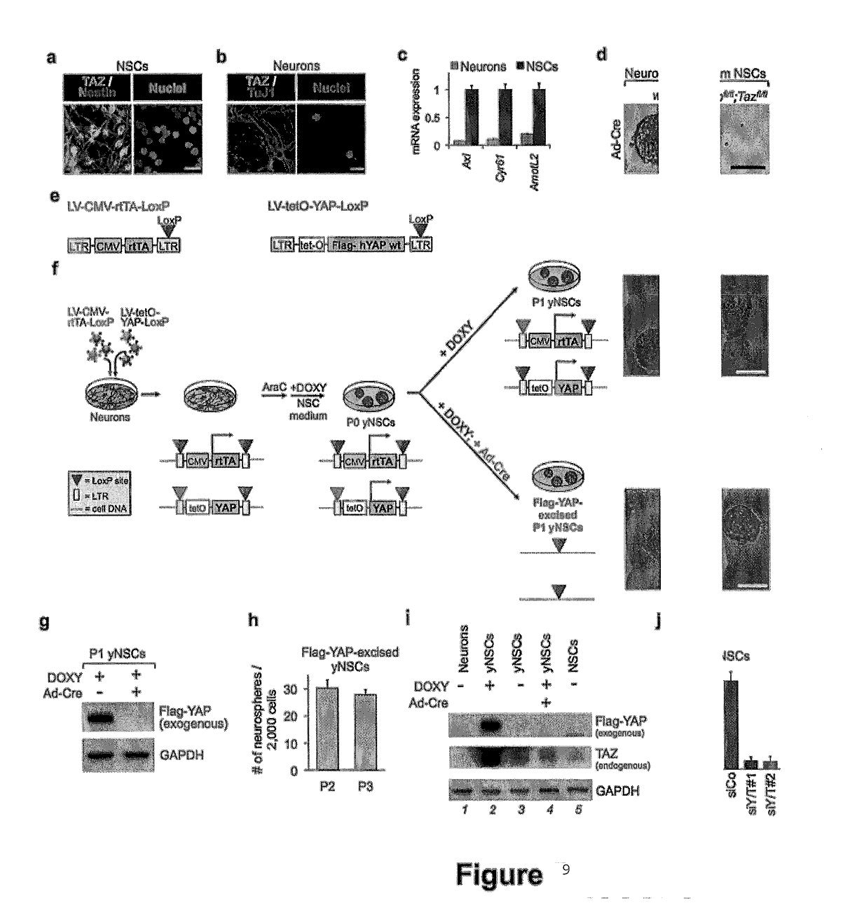

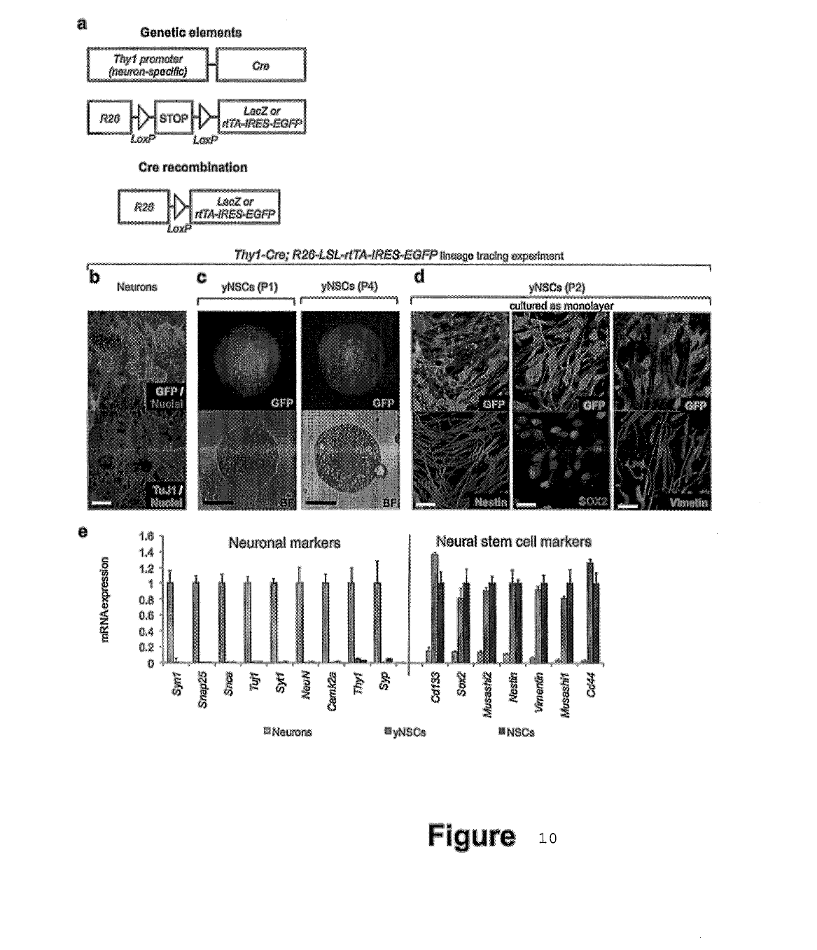

Neurons were prepared by dissociating the hippocampus or cortex of late mouse embryos (E19), and selected for post-mitotic neurons by culturing primary cells in neuronal-differentiation medium containing AraC for 4-7 days (Han, X. J. et al. CaM kinase I alpha-induced phosphorylation of Drp1 regulates mitochondrial morphology. The Journal of cell biology 182, 573-585 (2008)). This procedure eliminates proliferating cells, resulting in a population of mature post-mitotic neurons (>95%) displaying multiple neurites and expressing βIII-Tubulin (TuJ1), NeuN and other typical neuronal markers (see below and FIG. 10e). In parallel, primary neural SCs (NSCs) from dissociated cerebral hemispheres have also been derived; as previously reported (Palmer, T. D., Takahashi, J. & Gage, F. H. The adult rat hippocampus contains primordial neural stem cells. Molecular and cellular neurosciences 8, 389-404 (1997)), NSCs formed floating neurospheres that could be passaged multiple times. Then, by immunofluorescence, the expression of YAP or TAZ in neurons and NSCs was compared. Endogenous YAP and TAZ proteins were highly expressed and nuclearly localized in NSCs, but absent in neurons (FIG. 3a, b, and FIG. 9a, b); consistently, YAP/TAZ target genes are specifically upregulated in NSCs (FIG. 9c). Importantly, endogenous YAP/TAZ are essential to sustain the expansion of primary NSCs in vitro, as ex-vivo Adeno-Cre-mediated deletion of YAP/TAZ from Yapfl/fl; Tazfl/fl NSCs blunted neurosphere formation (FIG. 9d).

It has also been tested whether ectopic expression of YAP/TAZ in neurons was sufficient to convert them into NSCs. For this, primary cells were infected with lentiviral vectors encoding for rtTA and inducible wild-type YAP (see Methods). After AraC, neurons were shifted to NSC medium in the presence of doxycycline (see experimental outline in FIG. 3c). Remarkably, after 2 weeks, neurospheres-like structures emerged from YAP-expressing neurons, but never from neurons transduced with rtTA alone, or rtTA combined with EGFP, empty vectors or transcriptionally inactive YAPS94A. These “P0” spheres were then transferred to new plates for further growth, and could be then propagated for several passages as clonal outgrowths after single cell dissociation, indistinguishably from parental NSCs. Of note, the propagation of YAP-induced neurospheres did not require addition of doxycycline (FIG. 3d-g), indicating that transient exposure to exogenous YAP is sufficient to induce self-renewal properties that are autonomously maintained. In line, as shown by experiments with a Cre-excisable tetO-YAP lentiviral vector, the whole YAP encoding viral cassette could be deleted without effects on yNSC maintenance (FIG. 9 e-h). It has further been established that the self-renewal properties of yNSCs are actually sustained by reactivation of endogenous YAP/TAZ. Two lines of evidence support this conclusion: first, endogenous TAZ is induced in yNSCs and remains as such after doxycycline withdrawal and Cre-mediated excision of tetO-YAP cassette (FIG. 9i); second, YAP/TAZ depletion in yNSCs by transfecting independent pairs of siRNAs greatly impairs their self-renewal properties (FIG. 9j). These results raise an interesting parallel between the requirement of YAP/TAZ in native NSCs and induced yNSCs.

To validate that the NSC-like cells were indeed derived from terminally differentiated neurons, YAP-induced reprogramming in genetically lineage-traced neurons has been repeated. For this, mice carrying the established neuronal driver Thy1-Cre20 and the R26-LSL-LacZ reporter have been used (see scheme in FIG. 10a). It could be confirmed that at least a fraction of hippocampal neurons derived from Thy1-Cre; R26-LSL-LacZ mice were indeed β-gal positive, whereas NSCs derived from the same strain were always β-gal negative, thus confirming that Thy1-Cre is not active in SCs (FIG. 3h). Upon YAP-induced reprogramming, lineage-traced neurons gave rise to β-gal-positive neurospheres (FIG. 3h), indicating that YAP-induced NSCs (or yNSCs) indeed originated from differentiated neurons rather than through amplification of pre-existing, contaminating progenitors. Similar results were obtained with an independent lineage tracing experiment involving Thy1-Cre and a different reporter, R26-LSL-rtTA-IRES-EGFP. In this set up, only Thy-1-traced neurons express not only EGFP but also rtTA, ensuring that reprogramming by exogenous tetO-YAP occurs only in differentiated cells (see scheme in FIG. 10a). Infection of these neurons with tetO-YAP indeed generated EGFP-positive neurospheres (FIG. 10 b-d), while infection with empty tetO vector or transcriptionally-defective YAP was inconsequential. As additional controls, infection of neurons from R26-LSL-rtTA-IRES-EGFP (i.e., from littermates lacking Thy1-Cre) also did not result in any yNSCs. Similarly, yNSCs were obtained with a related but independent reprogramming strategy from neurons explanted from Syn1-Cre; R26-LSL-rtTA-IRES-EGFPmice, in which only synapsin-positive, differentiated neurons express rtTA21 and can be thus reprogrammed by tetO-YAP into EGFP-positive yNSCs (FIG. 10e-f). The changes in morphology occurring over reprogramming of neurons explanted from Syn1-Cre; R26-CAG-LSL-tdTomato mice after infection with rtTA and tetO-YAP have also been followed. After doxycycline addiction, tdTomato-traced neurons lost neurites in few days, and, within 10 days, adopted a flattened/elongated morphology and started proliferating, ultimately generating tdTomato-positive neurosphere-like structures.

Next, yNSCs have been characterized by immunofluorescence and marker gene expression. As shown in FIG. 3i and FIG. 10e, yNSCs have completely lost expression of the terminal differentiation markers present in the original hippocampal neurons (such as Tuj1, Tau and NeuN), and instead express high levels of NSC markers (such as Nestin, Sox2, Vimentin), and to level comparable to native NSCs. The use of Thy-1-Cre; R26-LSL-rtTA-IRES-EGFP lineage-traced neurons confirmed the origin of nestin-, Sox2-, Vimentin-positive yNSCs from converted differentiated cells (FIG. 10d). Collectively, the above experiments indicate that expression of YAP endows NSC-like characteristics to neurons.

In order to characterize to what extent YAP triggers neuronal conversion to a bona-fide NSC-status, the transcriptome of parental neurons, yNSCs and control NSCs have been compared. As shown in FIG. 3j, yNSCs completely lost their neuronal identity and acquired a gene expression profile closely similar to native NSCs. By Gene Ontology (GO), genes upregulated in both yNSCs and NSCs were specifically enriched of gene categories associated to positive regulation of the cell cycle and development/maintenance of the neural progenitor state. Genes downregulated in both yNSCs and NSCs were specifically enriched for GO terms related to terminal differentiation of neurons, transmission of nerve impulse, and nerve cell function.

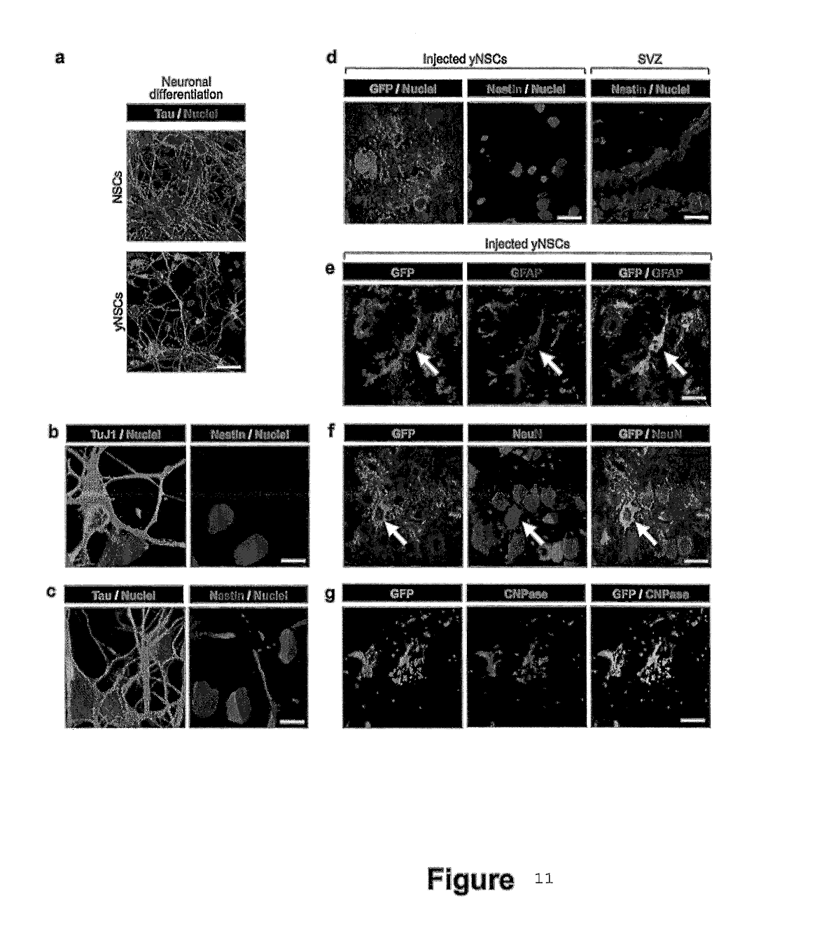

Neural SCs are defined as tripotent, as defined by their ability to differentiate in astrocytes, neurons and oligodendrocytes. The developmental potential of yNSCs was thus examined and compared to NSCs. yNSCs plated on fibronectin and treated with BMP4 and LIF22 completely switched to a typical astrocyte morphology, also expressing high levels of GFAP (FIG. 3k). For neuronal differentiation, a recently reported culture system involving plating of NSCs on Matrigel has been implemented (Choi, S. H. et al. A three-dimensional human neural cell culture model of Alzheimer's disease. Nature 515, 274-278 (2014)) (see Methods). Under these conditions, yNSCs, underwent widespread differentiation in Nestin-negative, TuJ1- and Tau-positive neurons characterized by an extensive neurite outgrowth (FIG. 3l, FIG. 11a-c). Moreover, upon treatment with IGF and T324, yNSCs could also differentiated into oligodendrocytes, displaying the typical branching morphology and CNPase positivity (FIG. 3m). After transplantation into the brain of newborn mice (n=5), EGFP-labeled yNSCs readily lost Nestin positivity and differentiated intoastrocytes, neurons and oligodendrocytes within the brain parenchyma (FIG. 11d-g). No tumor formation was observed after histological examination. Thus, YAP induces conversion of neurons to a NSC-like status.

### Example 4: Ex Vivo Generation of Pancreatic Progenitors from Exocrine Cells

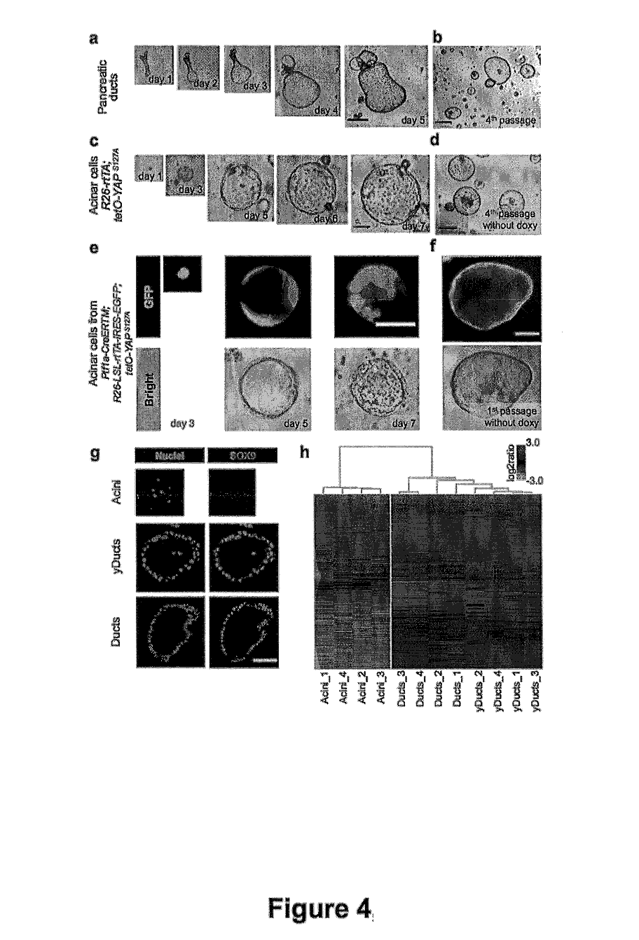

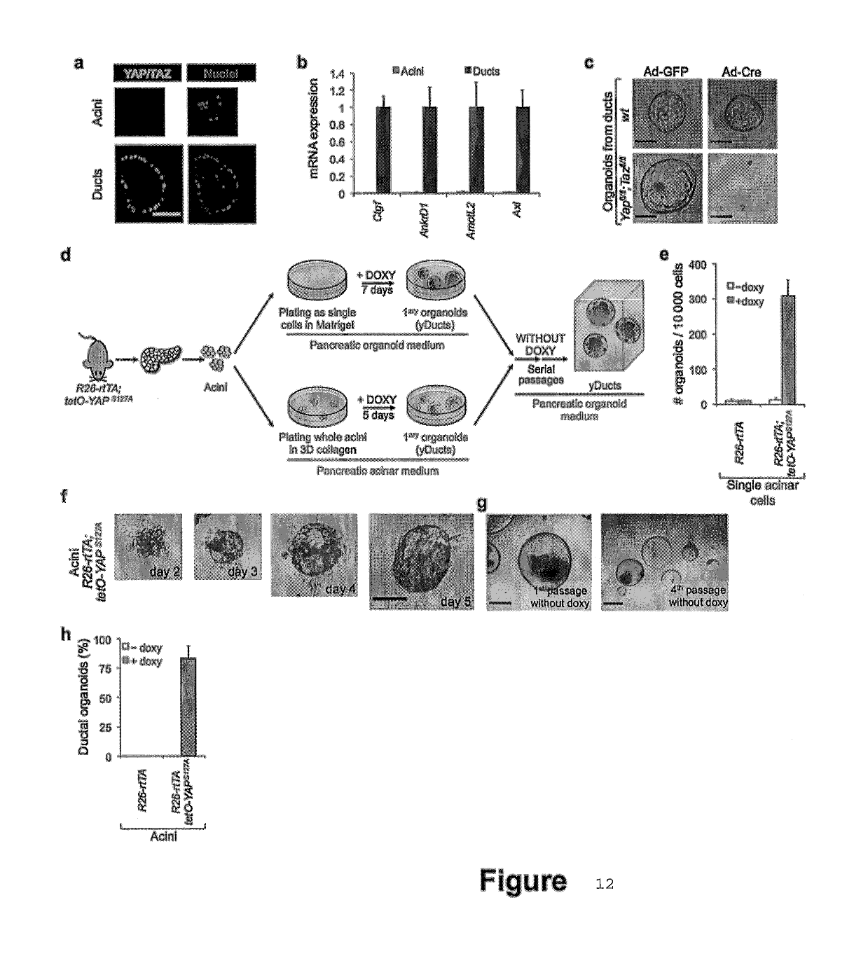

Pancreatic progenitors purified from the pancreatic duct have been recently shown to be expandable in vitro as organoids (Huch, M. et al. Unlimited in vitro expansion of adult bi-potent pancreas progenitors through the Lgr5/R-spondin axis. The EMBO journal 32, 2708-2721 (2013)) (FIG. 4a,b). Similarly to MaSCs and NSCs, pancreatic progenitors display nuclear and transcriptionally active YAP/TAZ, and genetically require YAP/TAZ for propagationas organoids (FIG. 12a-c). Since pancreatic progenitors are rare in the normal pancreas, it may be assumed that acinar cells, at least in principle, could represent a potential alternative source of autologous progenitors, as they are abundant and during injury or inflammation, have been shown to undergo ductal metaplasia (Puri, S., Folias, A. E. & Hebrok, M. Plasticity and Dedifferentiation within the Pancreas: Development, Homeostasis, and Disease. Cell Stem Cell (2014)).

Aiming to exploit this fate plasticity, it was then checked whether YAP expression could convert explanted primary acinar cells, normally void of endogenous YAP/TAZ, into ductal progenitors in vitro. To this end, pancreatic acini from R26-rtTA; tetOYAPS127A adult mice were isolated and dissociated to obtain a cell preparation highly enriched in exocrine cells (>400 fold, see Methods). Cells were plated in 100% Matrigel and added of doxycycline in pancreas organoid medium (see scheme in FIG. 12d). In just few days, acinar cells induced to express YAP, but not those left without doxycycline, expanded as cyst-like organoids (FIG. 4c,d, and FIG. 12e). Acinar cells derived from control R26+/rtTA mice remained as single cells or, more rarely, formed tiny cysts, but never organoids (FIG. 12e). After initial derivation, YAP-induced organoids (or “yDucts”) could be passaged for several months even in absence of doxycycline (for at least 10 passages, 6 months, see FIG. 12d). Individual organoids could be manually picked and expanded as clonal lines. By morphology, size and growth pattern, organoids derived from converted acinar cells were comparable to those obtained from handpicked pancreatic duct fragments after whole pancreas dissociation (Huch, M. et al. Unlimited in vitro expansion of adult bi-potent pancreas progenitors through the Lgr5/R-spondin axis. The EMBO journal 32, 2708-2721 (2013)) (FIG. 4b,d).

As an alternative strategy (avoiding primary acinar cells the harsh treatment of single cell dissociation with trypsin), whole pancreatic acini explanted from R26-rtTA; tetOYAPS127A have been embedded in collagen and cultured in low serum, that is, conditions that have been shown to preserve acinar cell identity ex-vivo (Means, A. L. et al. Pancreatic epithelial plasticity mediated by acinar cell transdifferentiation and generation of nestin-positive intermediates. Development 132, 3767-3776 (2005)) (see also experimental outline in FIG. 12d). When treated with doxycycline to induce YAP expression, pancreatic acini converted within few days to ductal organoid structures, and with extremely high efficiency (>70%) (FIG. 12 f-h). As control, acini lacking YAP expression (e.g., left without doxycycline, see FIG. 12h), remained as such for over 2 weeks and never converted to ducts. After transferring to 100% Matrigel/pancreatic organoid medium (a step involving mechanical dissociation), the YAP-induced ducts, but not control acini, regrew into organoids and could be maintained for several passages after single cell dissociation even in absence of doxycycline (FIG. 12g).

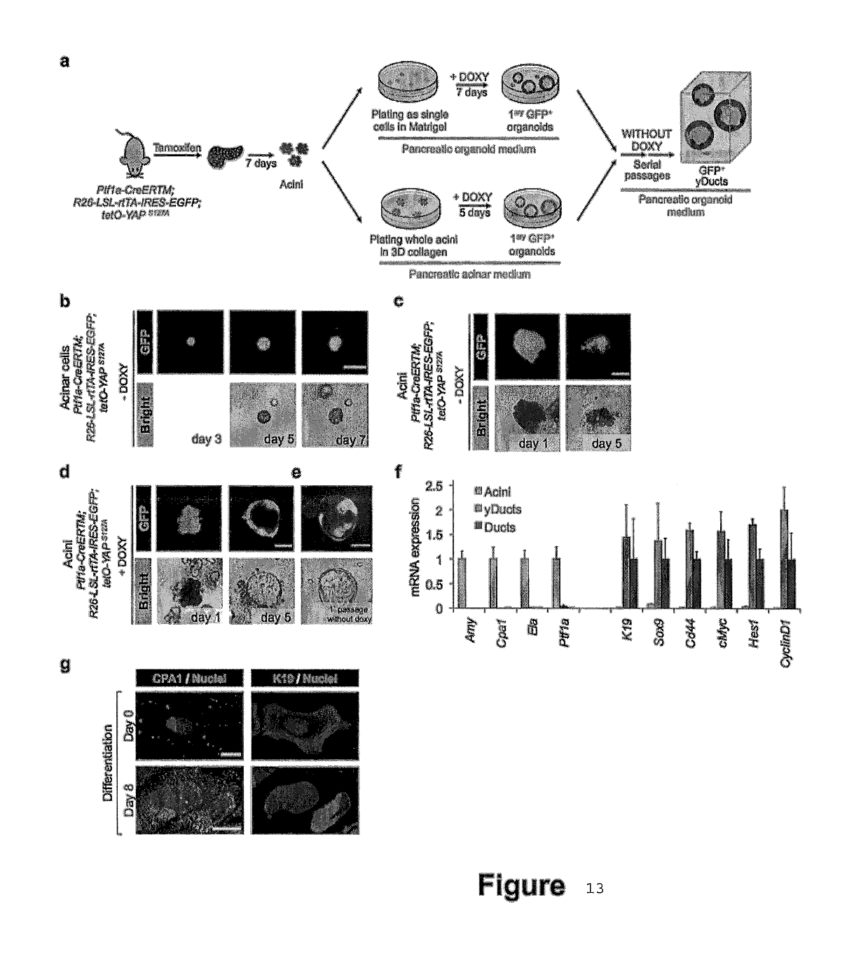

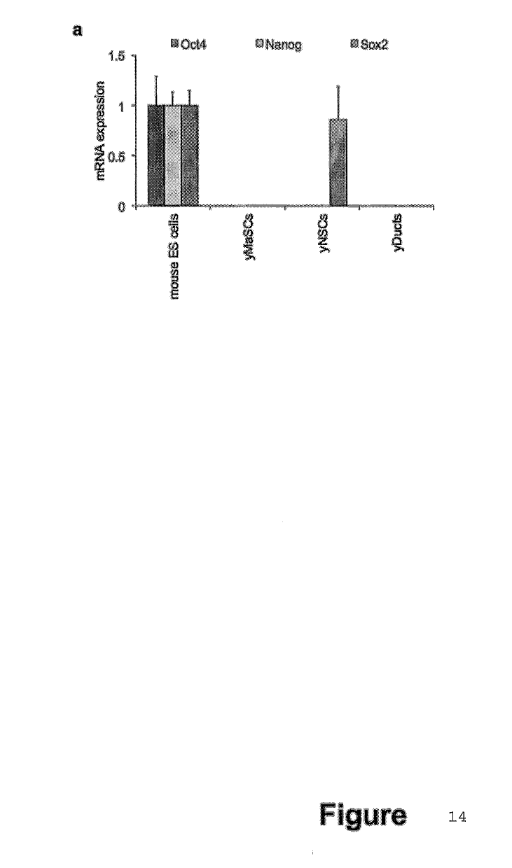

To validate that yDucts were indeed derived from differentiated exocrine acinar cells, genetic lineage tracing experiments were carried out using Ptf1a-CreERTM; R26-LSL-rtTA-IRES-EGFP; tetO-YAP S127A mice. It has been previously shown that, in the Ptf1a-CreERTM; R26-LSL-rtTA-IRES-EGFP background, tamoxifen treatment of adult mice causes irreversible genetic tracing of pancreatic acinar cells exclusively (Pan, F. C. et al. Spatiotemporal patterns of multipotentiality in Ptf1a-expressing cells during pancreas organogenesis and injury-induced facultative restoration. Development 140, 751-764 (2013)). One-week post-tamoxifen, pancreata were explanted to prepare whole acini or single-cell dissociated acinar cells, that were cultured as above (see experimental outline in FIG. 13a). These were EGFP-positive and, in absence of doxycycline, never formed any organoid FIG. 13b,c). Instead, doxycycline-induced YAP expression caused formation of expandable yDucts that retained EGFP positivity over passaging, formally demonstrating their derivation from terminally differentiated exocrine cells (FIG. 4e,f and FIG. 13d,e).

In section, organoids appeared as epithelial monolayers surrounding a central cavity (FIG. 4g). By qRT-PCR and immunofluorescence, organoids lost markers of exocrine differentiation (Ptf1a, a-amylase, elastase, and CPA1) and acquired expression of ductal markers (K19, Sox9, Hes1, Cd44), proliferative markers (cMyc and cyclinD1) all to levels comparable to those of native ductal organoids (FIG. 4g and FIG. 13f). To determine the extent of YAP-induced conversion of acinar cells, and their molecular overlap with native ductal progenitors, we carried out transcriptomic analyses. As shown in FIG. 4h, yDucts diverged profoundly from parental acinar cells to become overtly similar to bona-fide pancreatic progenitors. Under differentiating conditions, yDuct-derived cells could be induced to re-express the differentiated exocrine marker CPA1 and to downregulate K19 (FIG. 13g). When transplanted within a drop of Matrigel into the pancreas of NOD-SCID mice, yDucts remained as such, and never formed any tumor (n=6, data not shown) at least in the time-frame of the experiment carried out herein (6/7 weeks), confirming that yDucts are non-transformed. Together, the results indicate that exocrine cells with a history of exposure to YAP acquired key molecular and biological features of ductal pancreatic progenitors.

The present invention shows for the first time that expression of a single factor, YAP, into terminally differentiated cells explanted from different tissues efficiently creates cells with functional and molecular attributes of their corresponding tissue-specific SCs, that can be expanded ex-vivo as organoid cultures. The ySC state can be transmitted through cell generations without need of continuous expression of ectopic YAP/TAZ, indicating that a transient activation of ectopic YAP or TAZ is sufficient to induce a heritable self-renewing state.

According to the present invention, YAP/TAZ proteins are presented at the centerpiece of the somatic SC state whenever natural, pathological or ex-vivo conditions demand de novo generation and expansion of resident or facultative SCs.

The generation of autologous induced-SCs from various tissues by YAP/TAZ according to the present invention also holds the possibility to investigate somatic stemness or to expand rare cells, particularly in conditions in which aging or diseases have exhausted the endogenous SC pool. Finally, the present invention also raise the prospects to boost the body's regenerative capacity by sustaining YAP/TAZ expression at injury sites or as transplanted “super-SCs” able to produce new and more functional tissues than regular SCs.

### Methods

### Reagents, Plasmids and Transfections

Doxycycline hyclate, fibronectin, collagen I, heparin, insulin, dexamethasone, SBTI (Soybean Trypsin Inhibitor), gastrin, N-acethylcysteine, nicotinamide, T3 (Triiodo-L-Thyronine), tamoxifen and 4-OH-tamoxifen were from Sigma. Murine EGF, murine bFGF, human FGF10, human Noggin, human IGF, murine prolactin and BMP4 were from Peprotech. N2, B27, BPE and ITS-X (Insulin-Transferrin-Selenium-Ethanolamine) supplements were from Life Technologies. R-Spondin1 was from Sino Biological. Matrigel was from BD Biosciences (Corning). Rat tail collagen type I was from Cultrex. GFP- and Cre-expressing adenoviruses were from University of Iowa, Gene Transfer Vector Core. For inducible expression of YAP and TAZ, cDNA for siRNA-insensitive Flag-hYAP1 wt, S94A (TEAD-binding mutant, Zhao, B. et al. TEAD mediates YAP-dependent gene induction and growth control. Genes & development 22, 1962-1971 (2008))) and 5SA (LATS-mutant sites)(Aragona, M. et al. A mechanical checkpoint controls multicellular growth through YAP/TAZ regulation by actin-processing factors. Cell 154, 1047-1059 (2013)) and for Flag-mTAZ4SA (Azzolin, L. et al. Role of TAZ as mediator of Wnt signaling; Cell 151, 1443-1456 (2012)) were subcloned in FUW-tetO-MCS, obtained by substituting the Oct4 sequence in FUW-tetO-hOct4 (Addgene #20726 (Hockemeyer, D. et al. A drug-inducible system for direct reprogramming of human somatic cells to pluripotency; Cell Stem Cell 3, 346-353 (2008)) with a new multiple cloning site (MCS). This generated the FUW-tetO-wtYAP, FUW-tetO-YAPS94A, FUW-tetO-YAP5SA, FUW-tetO-TAZ4SA used throughout this study. FUW-tetO-MCS (empty vector) or FUW-tetO-EGFP plasmids were used as controls, as previously indicated⁸. All available in Addgene as #.

For stable expression of GFP, we used pRRLSIN.cPPT.PGK-GFP.WPRE (gift of L. Naldini) lentiviral vector.

For Cre-excisable expression of rtTA, we used LV-CMV-rtTA-LoxP (see scheme Extended Data FIG. 5e), obtained by substituting the Cre cDNA in LV-CMV-Cre-LoxP with the cDNA of rtTA from FUdeltaGW-rtTA (Addgene #19780 (Maherali, N. et al. A high-efficiency system for the generation and study of human induced pluripotent stem cells. Cell Stem Cell 3, 340-345 (2008).)). Available in Addgene as #.

For Cre-excisable lentiviral vector containing the tetO-Flag-hYAP wt cassette, we used LV-tetO-YAP wt-LoxP (see scheme FIG. 9e), obtained by substituting the CMV-Cre cassette in LV-CMV-Cre-LoxP with the tetO-Flag-hYAP wt cassette from FUW-tetO-Flag-hYAP1 wt. Available in Addgene as #.

All constructs were confirmed by sequencing.

siRNA transfections were done with Lipofectamine RNAi-MAX (Life technologies) in antibiotics-free medium according to manufacturer instructions. Sequences of siRNAs targeting murine Yap and Taz are as previously described (zzolin, L. et al. YAP/TAZ incorporation in the beta-catenin destruction complex orchestrates the Wnt response. Cell 158, 157-170 (2014)).

DNA transfections were done with TransitLT1 (Minis Bio) according to manufacturer instructions.

### Lentivirus Preparation

Lentiviral particles were prepared by transiently transfecting with TransIT-LT1 in Opti-MEM lentiviral vectors (10 micrograms/10 cm dishes) together with packaging vectors pMD2-VSVG (2.5 micrograms) and pPAX2 (7.5 micrograms) in HEK293T cells (checked routinely for absence of mycoplasma contaminations). Virus-producing HEK293T cells were cultured in DMEM (Life Technologies), supplemented with 10% FBS, glutamine and antibiotics. Supernatants were collected 48 hours post-transfection and lentiviral titer was determined using the QuickTiter Lentivirus Titer kit (lentivirus-associated HIV p24; Cell Biolabs) according to the manufacturer's protocol. The collected supernatant were filtered through 0.45 micrometers and directly stored at −20° C.; we did not concentrate viral supernatants. Each viral supernatant was used at a final titer of about 2-5 ng of p24/ml (see specifics below). In our hands, this typically corresponds to a simple 1:4 dilution of the each viral supernatant, in turn corresponding to a working final viral particle concentration of about 5×10⁷ particles/ml. As determined by PCR of integrated lentiviral DNA of HEK293T transduced with pRRL-EGFP, this roughly corresponds to 5×10⁵ transduction units (TU)/ml.

Primary Mammary Epithelial Cells (MECs) Isolation and Induction of yMaSCs

Primary MECs were isolated from the mammary glands of 8- to 12-week-old virgin C57BL/6J mice (unless otherwise specified), according to standard procedures. Mammary glands were minced and then digested with 6000 U/ml collagenase I (Life Technologies) and 2000 U/ml hyaluronidase (Sigma) in the DMEM/F12 (Life Technologies) at 37° C. for 1 hour with vigorous shaking. The digested samples were pipetted, spun down at 1500 rpm for 5 min, and incubated 3 min in 0.64% buffered NH₄Cl (Sigma) in order to eliminate contaminating red blood cells. After washing with DMEM/F12+5% FBS, cells were plated for 1 hour at 37° C. in DMEM/F12+5% FBS: in this way, the majority of fibroblasts attached to the tissue culture plastic, whereas mammary epithelial populations did not and were therefore recovered in the supernatant. After washing in PBS/EDTA 0.02%, MECs were further digested with 0.25% trypsin (Life Technologies) for 5 min and 5 mg/ml dispase (Sigma) plus 100 μg/ml DNase I (Roche) for other 10 min. The digested cells were diluted in DMEM/F12+5% FBS and filtered through 40 μm cell strainers to obtain single cell suspensions cells and washed once in the same medium.

For separating various MEC subpopulations cells were stained for 30 min at 4° C. with antibodies against CD49f (PE-Cy5, cat. 551129, BD Biosciences), CD29 (PE-Cy7, cat. 102222, BioLegend), CD61 (PE, cat. 553347, BD Biosciences), EpCAM (FITC, cat. 118208, BioLegend) and lineage markers (APC mouse Lineage Antibody Cocktail, cat. 51-9003632, BD Biosciences) in DMEM/F12.

The stained cells were then resuspended in PBD/BSA 0.1% and sorted on a BD FACS Aria sorter (BD Biosciences) into luminal differentiated (LD) cells, luminal progenitor (LP) cells and mammary stem cells (MaSCs).

Primary sorted subpopulations from FACS were plated on collagen I-coated supports and cultured in 2D in mammary (MG) medium (DMEM/F12 supplemented with glutamine, antibiotics, 10 ng/ml murine EGF, 10 ng/ml murine bFGF, and 4 μg/ml heparin with 2% FBS).

For induction of yMaSCs, adherent luminal differentiated cells were transduced for 48 hours with FUW-tetO-YAP, or FUW-tetO-TAZ, in combination with rtTA-encoding lentiviruses. As a (negative) control, LD cells were transduced with FUW-tetO-EGFP (FIG. 1e-f and FIG. 6c) in combination with rtTA-encoding lentiviruses. Each viral supernatant was used at a final titer of about 4-5 ng of p24/ml (see above the paragraph lentivirus preparation). After infection, adherent cells were washed and treated with 2 μg/ml doxycycline for 7 days in MG medium for activating tetracycline-inducible gene expression (see scheme in FIG. 1d) to obtain “yMaSCs”. After doxycycline treatment for 7 days in 2D culture, yMaSCs were processed for further assays or analysis. Unless otherwise specified, yMaSCs were generated from wild-type YAP (FUW-tetO-wtYAP, Addgene #).

For the experiment depicted in FIG. 2b and FIG. 7d,e we obtained LD cells from K8-CreERT2; R26-LSL-YFP/+ virgin female mice. These cells were plated and after attachment they were treated with 1 μM 4 OH-Tamoxifen for 24 hours. Cells were then transduced for 48 hours with FUW-tetO-wtYAP in combination with stable rtTA-encoding lentiviral supernatant. Negative control cells were provided by LD cells transduced with FUW-tetO-MCS (empty vector) in combination with rtTA-encoding lentiviral supernatants. Each viral supernatant was used at a final titer of about 4-5 ng of p24/ml. After infection, cells were washed, treated with doxycycline in MG medium and treated as the others (see below).

### Matrigel Culture of Mammary Colonies and Organoids

After infection in 2D cultures and induction with doxycycline for 7 days, mammary cells were detached with trypsin and seeded at a density of 1,000 cells/well in 24-well ultralow attachment plates (Corning) in MG-colony medium (DMEM/F12 containing glutamine, antibiotics, 5% Matrigel, 5% FBS, 10 ng/ml murine EGF, 20 ng/ml murine bFGF, and 4 μg/ml heparin) containing doxycycline (2 μg/ml). Primary colonies were counted 14 days after seeding. To show the self-renewal capacity of yMaSCs independently of exogenous YAP/TAZ supply (i.e., independently of doxycycline administration), primary colonies were recovered from the MG-colony medium by collecting the samples and incubation in ice cold HBSS. Cells were dissociated and re-seeded in ultralow attachment plates in MG-colony medium without doxycycline for further passaging.

For mammary organoid formation, primary colonies were recovered from MG colony medium in cold HBSS and transferred in 100% Matrigel. After Matrigel formed a gel, MG organoid medium was added (Advanced DMEM/F12 supplemented with Hepes, GlutaMax, antibiotics, EGF, bFGF, heparin, noggin and R-Spondin1). Note that at this step we do not dissociate at single cell level the primary colonies but simply transfer them to organoid culture conditions. Also note that direct plating of MaSCs, LD control EGFP-infected, as well as YAP-infected cells, directly into organoid culture conditions did not result in any outgrowth, indicating that the intermediate step in colony culture conditions is required for organoid development. After few days, colonies started to form budding organoids. 2 weeks after seeding, organoids were removed from Matrigel as before, trypsin-dissociated and transferred to fresh Matrigel. Passages were performed in a 1:4-1:8 split ratio every 2 weeks for at least 9 months. For analysis, organoids were recovered from Matrigel as before, and either embedded in OCT medium (PolyFreeze, Sigma) to obtain frozen sections for immunofluorescence or processed for protein or RNA extraction. For α- and β-casein induction (FIG. 2f and FIG. 7i), Matrigel-embedded organoids derived from yMaSCs or MaSCs were treated with MG organoid medium supplemented with insulin (10 μg/ml) and dexamethasone (1 μg/ml) in the absence or presence of lactogenic hormone prolactin (5 μg/ml) for 7 days. Organoids were then recovered from Matrigel as before and processed for RNA extraction.

### Cleared Mammary Fat Pad Transplantation

For induction of yMaSCs meant for in vivo injection (FIG. 2h-1 and FIG. 8c,d), adherent luminal differentiated cells were transduced for 48 hours with FUW-tetO-wtYAP in combination with stable rtTA- and EGFP-encoding lentiviruses to trace with EGFP fluorescence the generation of transgenic mammary glands from yMaSCs. For this, we mixed in a 1:1:1 ratio the FUdeltaGW-rtTA, pRRL-CMV-GFP and the FUW-tetO-wtYAP viral supernatants each at a final viral titer of about 2.5-3.5 ng of p24/ml, and added an equal volume of serum-free MG medium with 2× concentrations of supplements. Negative control LD cells were transduced as above with FUW-tetO-EGFP, rtTA and pRRL-CMV-GFP. After infection, cells were treated as before (washed, induced with doxycycline for 7 days in MG medium) and then injected in the cleared fat pads (see below). For the experiment of FIG. 2 k,l, cells transduced in the same way were first cultured as clonal colonies and organoids. Organoids were then used for injection.

Cell aliquots resuspended in 10 μl PBS/10% Matrigel were injected into the inguinal mammary fat pads of NOD-SCID mice (Charles River), which had been cleared of endogenous mammary epithelium at 3 weeks of age. Animals were then administered doxycycline in the drinking water for 2 weeks and then maintained without doxycycline for additional 8-10 weeks. Transplanted mammary fat pads were examined for gland reconstitution by whole-mount staining, GFP native fluorescence and immunofluorescence on sections from paraffin-embedded biopsies. Only the presence of GFP-positive branched ductal trees with lobules and/or terminal end buds was scored as positive reconstitution. For whole-mount analysis of mammary glands, freshly-explanted glands were fixed in PFA 4% (2 hours) and ethanol 70% (overnight). Glands were rehydrated, stained overnight with hematoxylin, subsequently dehydrated in graded ethanols, cleared by incubation in benzyl-alcohol/benzyl benzoate (1:2; Sigma) and imaged.

Primary Neuron Isolation and Induction of yNSCs

Neurons were prepared from hippocampi or cortices of E18-19 embryos as previously described (Han, X. J. et al. CaM kinase I alpha-induced phosphorylation of Drp1 regulates mitochondrial morphology. The Journal of cell biology 182, 573-585 (2008)). Briefly, hippocampi and cortices were dissected under the microscope in ice cold HBSS as quick as possible, incubated with 0.05% trypsin (Life Technologies) 15 min at 37° C. and, after trypsin blocking, resuspended in DMEM/10% FBS supplemented with 0.1 mg/ml DNase I (Roche), and mechanically dissociated. Cells were then plated on poly-L-lysine-coated plates in DMEM (Life technologies) supplemented with 10% FBS, glutamine and antibiotics for hippocampal neurons or in DMEM/Neurobasal (1:1) supplemented with 5% FBS, 1× B27, glutamine and antibiotics for cortical neurons (day 1). After 24 hours (day 2), medium was changed to fresh DMEM/Neurobasal (1:1) supplemented with 5% FBS, 1× B27, glutamine and antibiotics and, when specified, the next day (day 3) cells were infected with FUW-tetO-wtYAP and FUdeltaGW-rtTA viral supernatants. Negative controls were provided by neurons transduced with FUdeltaGW-rtTA alone or in combination with FUW-tetO-EGFP or FUW-tetO-MCS (empty vector). Viral supernatants were used at a final titer of about 4-5 ng of p24/ml for FUdeltaGW-rtTA, and 2 ng of p24/ml for all other viruses (see above the paragraph lentivirus preparation). After 24 hour (day 4), cells were incubated in Neurobasal medium supplemented with 1× B27, glutamine, antibiotics, and 5 μM Ara-C (cytosine β-D-arabinofuranoside; Sigma) for additional 7 days at the end of which well-differentiated, complex network-forming neurons are visible. To induce yNSCs formation, treated neurons were switched to NSC medium (DMEM/F12 supplemented with 1× N2, 20 ng/ml murine EGF, 20 ng/ml murine bFGF, glutamine, and antibiotics) and 2 μg/ml doxycycline for activating tetracycline-inducible gene expression. After 7 days, half of this medium was substituted with fresh NSC medium containing 4 μg/ml doxycycline. Sphere formation was evident upon YAP induction after 10-14 days of doxycycline treatment.

Spheres were gently transferred into a 15 ml-plastic tube and let sediment (typically 10-15 min). After discarding the supernatant, spheres were transferred to new Petri dishes with fresh NSC medium without doxycycline and let grow for 3-4 additional days. These neurospheres were then dissociated to single cells with TrypLE Express (Life Technologies), resuspended in NSC medium without doxycycline and transferred to a new dish; this step was repeated for every passage, as for normal NSCs.

For the experiment depicted in FIG. 3h and Extended Data FIG. 6a, we obtained hippocampal neurons from Thy1-Cre; R26-LSL-LacZ/+ embryos (day 1 and 2 as above). These cells were transduced as above (day 3). Cells were then treated with AraC/B27 containing medium as before and, after 7 days, switched to doxycycline containing-NSC medium to activate YAP expression and induce yNSC formation from LacZ-positive neurons. Thy1-Cre; R26-LSL-LacZ/+ neurons transduced with FUdeltaGW-rtTA in combination with FUW-tetO-EGFP or FUW-tetO-YAPS94A never gave rise to any neurospheres. Each embryo genotype was confirmed on tail biopsies post-brain dissociation; as separate negative controls, neurons derived from R26-LSL-LacZ/+ littermates (Thy1-Cre negative) were transduced with FUW-tetO-wtYAP and FUdeltaGW-rtTA viral supernatants as above, and never gave rise to LacZ-positive yNSCs. These same neurons transduced with FUdeltaGW-rtTA in combination with FUW-tetO-EGFP or FUW-tetO-YAPS94A never gave rise to any neurospheres.

For the experiment depicted in FIG. 10a-d, we obtained hippocampal neurons from Thy1-Cre; R26-LSL-rtTA-IRES-EGFP/+ embryos (day 1 and 2 as above). These cells were transduced as above (day 3) with FUW-tetO-YAP wt alone (or FUW-tetO-empty vector or FUW-tetO-YAPS94A as negative controls). Cells were then treated with AraC/B27 containing medium as before and, after 7 days, switched to doxycycline containing-NSC medium to activate YAP expression and induce yNSC formation from GFP-positive neurons. Each embryo genotype was confirmed on tail biopsies post-brain dissociation; as separate negative controls, neurons derived from R26-LSL-rtTA-IRES-EGFP/+ littermates (Thy1-Cre negative) were treated as above, and never gave rise yNSCs.

For the experiment depicted in FIG. 10e-f, we obtained cortical neurons from Syn1-Cre; R26-LSL-rtTA-IRES-EGFP/+ embryos (day 1 and 2 as above). These cells were transduced as above (day 3) with FUW-tetO-YAP wt alone (or FUW-tetO-empty vector or FUW-tetO-YAPS94A as negative controls). Cells were then treated with AraC/B27 containing medium as before and, after 7 days, switched to doxycycline containing-NSC medium to activate YAP expression and induce yNSC formation from GFP-positive (synapsin-expressing) neurons.

For the experiment with excisable YAP vectors (FIG. 9 d-h), neurons were transduced (day 3) for 24 hours with LV-tetO-wtYAP-LoxP in combination with LV-CMV-rtTA-LoxP. Each viral supernatant was used at a final titer of 6 ng of p24/ml. Neurons were then treated with AraC/B27 containing medium as before and, after 7 days, switched to doxycycline containing-NSC medium to activate YAP expression.

### Primary Neural Stem Cells (NSCs) Isolation and Culture

Neural stem cells (NSCs) were isolated as previously reported (Ray, J. & Gage, F. H. Differential properties of adult rat and mouse brain-derived neural stem/progenitor cells. Molecular and cellular neurosciences 31, 560-573 (2006)) from the telencephalon of C57BL/6J E18 embryos or from mice of the indicated genotype. Telencephalons were minced and digested in trypsin 0.05% for 10 min at 37° C. The cell suspension was treated with DNaseI (Roche) and washed. NSCs were cultured in DMEM/F12 supplemented with N2, 20 ng/ml murine EGF, 20 ng/ml murine bFGF, glutamine and antibiotics. For passages, neurospheres were dissociated into single cells with TrypLE Express (Life Technologies).

### NSCs/yNSCs Transfection, Infection and Differentiation

Prior to transfection with siRNA, yNSCs were plated on fibronectin coated-plate in NSC medium, to allow a 2D culture; the next day, cells were transfected with siRNA and after 24 hours, replated in ultra-low attachment plates to allow neurosphere formation. Neurospheres were counted after 7 days from plating.

For adenoviral infection of wild-type (wt) or double Yap^{fl/fl}; Taz^{fl/fl} NSCs (FIG. 9d), single cells were plated in NSC medium containing adeno-Cre on ultra-low attachment plates and allowed to form neurospheres for 7 days.

For neuronal differentiation, NSCs or yNSCs were cultured over a thin Matrigel layer. Differentiation medium was Neurobasal supplemented with 1× B27, glutamine.

For astrocyte differentiation, NSCs or yNSCs were plated on fibronectin coated-plate in NSC medium, to allow a 2D culture. The next day, medium was changed to DMEM (Life Technologies) containing 25 ng/ml LIF, 25 ng/ml BMP4, glutamine, and antibiotics for 2 weeks.

For oligodendrocyte differentiation, NSCs or yNSCs were plated on fibronectin coated-plate in NSC medium, to allow a 2D culture. The next day, medium was changed to Neurobasal (Life Technologies) containing 1× B27, 500 ng/ml IGF, 30 ng/ml T3, glutamine, and antibiotics for 2 weeks.

### NSCs Transplantation

P0 CD1 mice pups were used for cell transplantations. Pups were anesthetized by hypothermia (3 minutes) and fixed on ice-cold block during cell injection. Cells were resuspended in ice-cold HBSS (5×10⁴ cells/μl) and injected into both hemispheres of neonatal mice with a 5 μl-volume Hamilton syringe (2 μl/injection). One month after the procedures, the grafted animals were perfused with PBS and 4% PFA, and the brains were excised and processed for immunofluorescence.

Pancreatic Acinar Cells Isolation and Induction of yDucts

Primary pancreatic acini were isolated from the pancreas of 6- to 9-week-old mice, according to standard procedures (Means, A. L. et al. Pancreatic epithelial plasticity mediated by acinar cell transdifferentiation and generation of nestin-positive intermediates. Development 132, 3767-3776 (2005)). Digested tissue was filtered through a 100 μm nylon cell strainer. The quality of isolated acinar tissue was checked under the microscope. For culture of entire acini, explants were seeded in neutralized rat tail collagen type I (Cultrex)/acinar culture medium (1:1) (Means, A. L. et al. Pancreatic epithelial plasticity mediated by acinar cell transdifferentiation and generation of nestin-positive intermediates. Development 132, 3767-3776 (2005)), overlaid with acinar culture medium (Waymouth's medium (Life Technologies) supplemented with 0.1% FBS (Life Technologies), 0.1% BSA, 0.2 mg/ml SBTI, 1×ITS-X (Life Technologies), 50 μg/ml BPE (Life Technologies), 1 μg/ml dexamethasone (Sigma), and antibiotics) once collagen formed a gel. For culture of isolated acinar cells, acini were further digested in 0.05% trypsin for 30 min at 37° C. to obtain a single cell suspension. Single acinar cells were plated in 100% Matrigel; once Matrigel formed a gel, cells were supplemented with pancreatic organoid medium (Advanced DMEM/F12 supplemented with 1× B27, 1.25 mM N-Acetylcysteine, 10 nM gastrin, 50 ng/ml murine EGF, 100 ng/ml human Noggin, 100 ng/ml human FGF10, 10 mM Nicotinamide, 1 μg/ml R-Spondin1 and antibiotics) supplemented with 0.2 mg/ml SBTI. To assess enrichment of acinar cells in our preparation, we compared RNA extracts from whole pancreas and our fresh acinar cell preparation for expression of exocrine cell markers, such as α-amylase, elastase and CPA1 (data not shown).

For induction of pancreatic organoids, entire acini or single acinar cells of the indicated genotypes cells were treated with 2 μg/ml doxycycline. Negative control cells were cultured in the same conditions in absence of doxycycline. Cells were treated with 2 μg/ml doxycycline for 7 days and organoid formation was morphologically followed. Organoids were then processed for further analyses.

For the experiment depicted in FIG. 4e-f (see also scheme in FIG. 13a), we obtained acinar explants form 6 week-old Ptf1a-CreERTM; R26-LSL-rtTA-IRES-EGFP/+; tetO-YAP^{S127A} mice (an, F. C. et al. Spatiotemporal patterns of multipotentiality in Ptf1a-expressing cells during pancreas organogenesis and injury-induced facultative restoration. Development 140, 751-764 (2013)). These mice were given tamoxifen by three daily i.p. injections of a 10 mg/ml solution in corn oil 1 week before pancreas dissociation in order to trace rtTA-IRES-EGFP⁺ exocrine acinar cells. Primary pancreatic acinar cells were isolated and cultured as above. For induction of pancreatic organoids, acinar explants were treated with 2 μg/ml doxycycline as above. As further negative control, Ptf1a-CreERTM; R26-LSL-rtTA-IRES-EGFP/+; tetO-YAP^{S127A} mice were administered with vehicle corn oil 1 week before pancreas dissociation and explanted acini were always EGFP negative and did not give rise to any organoids even upon doxycycline treatment (data not shown).

Matrigel Culture of yDucts Organoids

To show the self-renewal capacity of pancreatic organoids independently of exogenous YAP supply (i.e., independently of doxycycline administration), organoids were recovered from Matrigel or collagen cultures, trypsinized to obtain a single cell suspension and re-seeded in 100% Matrigel covered with pancreatic organoid medium. For analysis, organoids were recovered from Matrigel as before and processed for immunofluorescence or for protein or RNA extraction.

For the differentiation experiments shown in FIG. 13g, yDucts were removed from Matrigel, trypsin-dissociated and seeded as single cells in Matrigel-coated (1:50) chamber slides. Cells were expanded in DMEM supplemented with 0.5% BSA, 1% ITS-X and 1× N2 and 50 ng/ml EGF and antibiotics for 5 days. For differentiation, cells were switched to DMEM/F12 supplemented with 1% ITS-X, 10 ng/ml bFGF, 10 mM nicotinamide, 50 ng/ml Exendin-4 and 10 ng/ml BMP4 and antibiotics for further 8 days. Cells were fixed in 4% PFA at Day 0 or Day 8 of differentiation and processed for immunofluorescence.

### Culture of Pancreatic Ductal Organoids (Ducts)

For culture of pancreatic duct-derived organoids, pancreatic ducts were isolated from the bulk of the pancreas as previously described²⁵ with minor modifications. The whole pancreas of 6- to 9-week-old mice of the indicated genotypes was grossly minced and digested by collagenase/dispase dissociation: DMEM medium (Life Technologies) supplemented with collagenase type XI 0.012% (w/v) (Sigma), dispase 0.012% (w/v) (Life Technologies), 1% FBS (Life Technologies) and antibiotics at 37° C. for 1 hour. Isolated pancreatic duct fragments were hand-picked under a dissecting microscope, carefully washed in DMEM medium and embedded in 100% Matrigel. After Matrigel formed a gel, pancreatic organoid medium (Advanced DMEM/F12 supplemented with 1× B27, 1.25 mM N-Acetylcysteine, 10 nM gastrin, 50 ng/ml murine EGF, 100 ng/ml human Noggin, 100 ng/ml human FGF10, 10 mM Nicotinamide, 1 μg/ml R-Spondin1 and antibiotics) was added. Ductal fragments rapidly expanded to form cyst-like organoids within 5 days. Organoids were removed from Matrigel by incubation in ice cold HBSS, dissociated with trypsin 0.05% for 30 min to obtain a single cells suspension and reseeded in 100% fresh Matrigel. Organoid cultures were maintained for at least 9 months passaging every 10 days. For analysis, organoids were recovered from Matrigel as before and processed for immunofluorescence or for protein or RNA extraction.

For the experiment depicted in FIG. 12c, pancreatic duct fragments were isolated from 9 weeks old Yap^{fl/fl}; Taz^{fl/fl} mice, embedded in 100% Matrigel and cultured as above. Organoids were passaged once every 10 days. After at least 3 months of culture, organoids were removed from Matrigel by incubation in ice cold HBSS, trypsin-dissociated and transduced with adenovirus encoding for CRE recombinase to induce Yap/Taz knockout (or with GFP-encoding adenovirus as control). Single cells were resuspended in 2 ml Advanced DMEM/F12, transduced for 2 hours at 37° C. with adenovirus, washed in Advanced DMEM/F12 and seeded in 100% Matrigel. After Matrigel formed a gel, cells were maintained in pancreatic organoid medium and organoid formation capacity was morphologically monitored over a period of 10 days. Pancreatic ductal organoids obtained from wt mice were used as additional controls and treated as above.

### Immunofluorescence, Stainings and Microscopy

Immunofluorescences on PFA-fixed samples were performed as previously described (ordenonsi, M. et al. The Hippo transducer TAZ confers cancer stem cell-related traits on breast cancer cells. Cell 147, 759-772 (2011)). Briefly, samples were fixed 10 min at room temperature with 4% PFA solution. Slides were permeabilized 10 min at RT with PBS 0.3% Triton X-100, and processed for immunofluorescence according to the following conditions: blocking in 10% Goat Serum (GS) in PBS 0.1% Triton X-100 (PBST) for 1 hr followed by incubation with primary antibodies (diluted in 2% GS in PBST) overnight at 4° C., four washes in PBST and incubation with secondary antibodies (1:200 in 2% GS in PBST) for 2 hours at room temperature. Samples were counterstained with ProLong-DAPI (Molecular Probes, Life Technologies) to label cell nuclei.

For immunofluorescence on mammary organoids (FIG. 2b-e and FIG. 7c, e-h), organoids freshly recovered from Matrigel were embedded in OCT tissue-freezing medium (PolyFreeze, Sigma) and frozen on dry ice. 8 μm cryostat sections for all types of organoids were cut at −20° C. Sections were mounted on glass slides and dried for at least 30 min. The sections were then fixed with 4% formaldehyde for 10 min. After washing with PBS the sections were processed as described above. For immunofluorescence on pancreatic organoids or acini (FIG. 4g, and FIG. 12a), pancreatic acini and organoids were fixed overnight in PBS 4% PEA at 4° C., permeabilized with two washes in PBS 0.5% NP40 for 20 minutes at 4° C., followed by one wash in PBS 0.3% Triton X-100 for 20 minutes at room temperature. After two washes in PBS 0.1% Triton X-100 (PBST) for 15 minutes at room temperature, acini or organoids were blocked with two washes in PBST 10% GS for 1 hour at room temperature, and incubated overnight with primary antibodies. The following day, cells were washed twice in PBST 2% GS for 15 minutes at 4° C., and five more times in PBT 2% GS for 1 hour at 4° C. Secondary antibodies were incubated overnight. The third day, cells were washed five times in PBST for 15 minutes, incubated 20 min with DAPI solution and mounted in glycerol.

For immunofluorescence on mammary and brain tissue, biopsies were fixed with PFA, paraffin-embedded and cut in 10 μm-thick sections. Sections were re-hydrated and antigen retrieval was performed by incubation in citrate buffer 0.01 M pH 6 at 95° C. for 20 minutes. Slides were then permeabilized (10 min at RT with PBS 0.3% Triton X-100 for mammary sections and 10 min at RT with PBS 1% Triton X-100 for brain sections) and processed as described above.

Primary antibodies: anti-YAP (4912; 1:25) polyclonal antibody, anti-CNPase (5664S; 1:100) polyclonal antibody, anti-SOX2 (4900; 1:50) monoclonal antibody were from Cell Signaling Technology. anti-TAZ (anti-WWTR1, HPA007415; 1:25) polyclonal antibody, anti-α-SMA (A2547; 1:400) mouse monoclonal antibody and anti-amylase (A8273:1:200) rabbit polyclonal antibody were from Sigma. anti-TuJ1 (anti β-III-tubulin; MMS435P-100; 1:500) mouse monoclonal antibody was from Covance. anti-GFAP (Z0334; 1:1000) rabbit polyclonal antibody was from Dako. anti-Nestin (MAB353; 1:300) mouse monoclonal antibody and anti-Sox9 (AB5535; 1:200) rabbit polyclonal antibody were from Millipore. anti-E-cadherin (610181; 1:1000) monoclonal antibody was from BD Biosciences. anti-K14 (Ab7800; 1:100) mouse monoclonal antibody, anti-NeuN (Ab177487; 1:100) rabbit monoclonal antibody, anti-K8 (Ab14053; 1:100) chicken polyclonal antibody and anti-GFP (Ab13970; 1:100) polyclonal antibody were from Abcam. anti-GFP (A6455; 1:100) rabbit serum was from Life Technologies. anti-p63 (H137, sc-8343; 1:50) and anti-Vimentin (Vim C-20, sc-7557-R; 1:100) rabbit polyclonal antibodies were from Santa Cruz. anti-Tau (1:100) rabbit polyclonal antibody was from Axell. K19 was detected using the monoclonal rat anti-Troma-III antibody (DSHB; 1:50). Alexa-conjugated secondary antibodies (Life Technologies): Alexa-Fluor-488 donkey anti-mouse IgG (A21202); Alexa Fluor-568 goat anti-mouse IgG (A11031); Alexa-Fluor-647 donkey anti-mouse (A31571); Alexa Fluor-488 goat anti-mouse IgG₂ₐ (A21131), Alexa Fluor-647 goat anti-mouse IgG₁ (A21240), Alexa Fluor-488 donkey anti-rabbit IgG (A21206), Alexa-Fluor-568 goat anti-rabbit IgG (A11036), Alexa-Fluor-647 donkey anti-rabbit IgG (A31573); Alexa Fluor-555 goat anti-chicken IgG (A21437). Goat anti-rat Cy3 (112-165-167) was from Jackson Immunoresearch.

For X-gal staining (FIG. 3h), samples were permeabilized in PBS/NP-40 0.02%, fixed 1 hour in PFA 4% in PBS, washed twice in PBS/NP-40 0.02% and stained with the staining solution (X-gal (Sigma, B4252) 25 μg/ml, 4 mM potassium ferricyanide crystalline, 4 mM potassium ferricyanide trihydrate, 2 mM MgCl₂, 0.02% NP-40 in PBS).

Confocal images were obtained with a Leica TCS SP5 equipped with a CCD camera. Bright field and native-GFP images were obtained with a Leica DM IRB inverted microscope equipped with a CCD camera (Leica DFC 450C). Live cell imaging was performed with a A1Rsi+laser scanning confocal microscope (Nikon) equipped with NIS-Elements Advanced Research Software.

### Western Blot

Western blots were carried out according to standard procedures. Anti-YAP/TAZ (63.7; sc-101199) and anti-p63 (4A4; sc-8431) monoclonal antibodies were from Santa Cruz. anti-GAPDH (MAB347) monoclonal antibody was from Millipore. Anti-K14 (Ab7800) mouse monoclonal antibody and anti-K8 (Ab14053) chicken polyclonal antibody were from Abcam.

Quantitative Real-Time PCR (qRT-PCR)

Cells or tissues were harvested in TriPure (Roche) for total RNA extraction, and contaminant DNA was removed by DNase treatment. qRT-PCR analyses were carried out on retrotranscribed cDNAs with Rotor-Gene Q (Quiagen) thermal cycler and analyzed with Rotor-Gene Analysis6.1 software. Expression levels are always given relative to Gapdh, except for FIGS. 12b and 13f in which expression levels were normalized to 18-S rRNA. PCR oligo sequences for mouse samples are listed at the website http://www.bio.unipd.it/piccolo/protocols_and_tools.html.

### Microarray Experiments

For microarray experiments, Mouse Genome 430 2.0 arrays (Affymetrix, Santa Clara, Calif., USA) were used. Total RNA was extracted using TriPure (Roche) from:

1) luminal differentiated mammary cells (3 replicas), organoids derived from yMaSCs (3 replicas), and MaSCs (3 replicas);
2) cortical neurons (3 replicas), yNSCs (from YAP wild type-transduced cortical neurons, passage 2; 3 replicas), and native NSCs (3 replicas);
3) pancreatic exocrine acini (4 replicas), yDucts (passage 10; 4 replicas), and Ducts (passage 10; 4 replicas).

RNA quality and purity were assessed on the Agilent Bioanalyzer 2100 (Agilent Technologies, Waldbronn, Germany); RNA concentration was determined using the NanoDrop ND-1000 Spectrophotometer (NanoDrop Technologies Inc.). RNA was then treated with DNaseI (Ambion). In vitro transcription, hybridization and biotin labeling were performed according to Affymetrix 3′IVT protocol (Affymetrix). As control of effective gene modulation and of the whole procedure, we monitored the expression levels of tissue-specific markers of differentiated cells or stem/progenitors by qRT-PCR prior to microarray hybridization and in the final microarray data.

All data analyses were performed in R (version 3.1.2) using Bioconductor libraries (BioC 3.0) and R statistical packages. Probe level signals were converted to log 2 expression values using robust multi-array average procedure RMA⁴⁶ of Bioconductor affy package. Raw data are available at Gene Expression Omnibus under accession number GSE70174. Global unsupervised clustering was performed using the function hclust of R stats package with Pearson correlation as distance metric and average agglomeration method. Gene expression heatmaps have been generated using the function heatmap.2 of R gplots package after row-wise standardization of the expression values. Before unsupervised clustering, to reduce the effect of noise from non-varying genes, we removed those probe sets with a coefficient of variation smaller than the 90^{th} percentile of the coefficients of variation in the entire dataset. The filter retained 4511 probe sets that are more variable across samples in any of the 3 subsets (i.e., mammary, neuron, and pancreatic).

### Mice

C57BL/6J mice and NOD-SCID mice were purchased from Charles River. Transgenic lines used in the experiments were gently provided by: Duojia Pan (Zhang, N. et al. The Merlin/NF2 tumor suppressor functions through the YAP oncoprotein to regulate tissue homeostasis in mammals. Dev Cell 19, 27-38 (2010)) (Yap1^{fl/fl} and R26-LSL-LacZ); Cedric Blanpain (K8-CreERT2/R26-LSL-YFP) (Van Keymeulen, A. et al. Distinct stem cells contribute to mammary gland development and maintenance. Nature 479, 189-193 (2011)); Doron Merckler (Thy1-Cre)(Dewachter, I. et al. Neuronal deficiency of presenilin 1 inhibits amyloid plaque formation and corrects hippocampal long-term potentiation but not a cognitive defect of amyloid precursor protein [V717I] transgenic mice. The Journal of neuroscience: the official journal of the Society for Neuroscience 22, 3445-3453 (2002)); Ivan De Curtis (Syn1-Cre)(Zhu, Y. et al. Ablation of NF1 function in neurons induces abnormal development of cerebral cortex and reactive gliosis in the brain. Genes & development 15, 859-876 (2001)); Giorgio Carmignoto (R26-CAG-LSL-tdTomato) (Madisen, L. et al. A robust and high-throughput Cre reporting and characterization system for the whole mouse brain. Nature neuroscience 13, 133-140 (2010)); Fernando Camargo (tetO-YAP^{S127A}) (Camargo, F. D. et al. YAP1 increases organ size and expands undifferentiated progenitor cells. Curr Biol 17, 2054-2060 (2007)). Taz^{fl/fl} and double Yap^{fl/fl}; Taz^{fl/fl} conditional knock-out mice were as described in (Azzolin, L. et al. YAP/TAZ incorporation in the beta-catenin destruction complex orchestrates the Wnt response. Cell 158, 157-170 (2014)). Ptf1a-CreERTM (stock #019378), R26-LSL-rtTA-IRES-EGFP (stock #005670) and R26-rtTAM2 mice (stock #006965) were purchased from The Jackson Laboratory. Animals were genotyped with standard procedures and with the recommended set of primers. Animal experiments were performed adhering to our institutional guidelines as approved by CEASA.

To obtain Thy1-Cre; R26-LSL-LacZ/+ mice, we crossed Thy1-Cre hemizygous males with R26-LSL-LacZ/LSL-LacZ females. Littermate embryos derived from these crossings were harvested at E18-19 and kept separate for neurons/NSCs derivation; genotypes were confirmed on embryonic tail biopsies.

To obtain Thy1-Cre; R26-LSL-rtTA-IRES-EGFP/+ mice, we crossed Thy1-Cre hemizygous males with R26-LSL-rtTA-IRES-EGFP/LSL-rtTA-IRES-EGFP females. Littermate embryos derived from these crossings were harvested at E18-19 and kept separate for neurons derivation; genotypes were confirmed on embryonic tail biopsies.

To obtain Syn1-Cre lineage tracing studies, we used Syn1-Cre hemizygous females (as transgene expression in male mice results in germline recombination (Rempe, D. et al. Synapsin I Cre transgene expression in male mice produces germline recombination in progeny. Genesis 44, 44-49 (2006))) with R26-LSL-rtTA-IRES-EGFP homozygous males or R26-CAG-LSL-tdTomato/+ males. Littermate embryos derived from these crossings were harvested at E18-19 and kept separate for neurons derivation; genotypes were confirmed on embryonic tail biopsies.

To obtain R26-rtTAM2; tetO-YAP^{S127A} mice, we crossed R26-rtTAM2/+ mice with tetO-YAP^{S127A} mice. R26-rtTAM2/+ littermates were used as negative control.

To obtain Ptf1a-CreERTM; R26-LSL-rtTA-IRES-EGFP/+; tetO-YAP^{S127A} mice, we crossed Ptf1a-CreERTM; R26-LSL-rtTA-IRES-EGFP/LSL-rtTA-IRES-EGFP mice with tetO-YAP^{S127A} mice. Ptf1a-CreERTM; R26-LSL-rtTA-IRES-EGFP/+ littermates were used as negative control.

### Statistics

The number of biological and technical replicates and the number of animals are indicated in Fig. legends and specification. All tested animals were included. Animal ages are specified in the specification. Sample size was not predetermined. Experiments were performed without methods of randomization or blinding. For all experiments with error bars the standard deviation (s.d.) was calculated to indicate the variation within each experiment.

1. A method for generating somatic stem cells, comprising the steps of:
a. providing at least one differentiated cell, committed progenitor or partially differentiated cell;
b. inducing an increased expression or activity of a YAP protein, and/or a TAZ protein, and/or a functional fragment of the YAP and/or the TAZ protein, and/or an activated version of the YAP and/or the TAZ protein, or derivatives thereof in at least one differentiated cell or committed progenitor or partially differentiated cell;
c. generating a somatic stem cell out of said differentiated cell, committed progenitor or partially differentiated cell.

2. The method according to claim 1, wherein expression or activation of said YAP/TAZ protein, and/or said functional fragment, and/or said activated version, and/or said derivative thereof in at least one differentiated cell, committed progenitor or partially differentiated cell is increased transiently.

3. The method according to claim 2, wherein expression of said YAP/TAZ protein, and/or said functional fragment, and/or said activated version, and/or said derivative thereof in at least one differentiated cell, committed progenitor or partially differentiated cell is increased ectopically.

4. The method according to claim 2, wherein said YAP/TAZ protein is endogenous.

5. The method according to claim 4, wherein the activity of said endogenous YAP/TAZ protein is increased by influencing a biological activity of said endogenous YAP/TAZ protein, and/or by influencing a cellular stability of said endogenous YAP/TAZ protein, and/or by a influencing a cellular localization of said endogenous YAP/TAZ protein.

6. The method according to claim 3, further comprising the step of transfecting said at least one differentiated cell of step a) with a vector comprising a nucleotide sequence coding for a wild-type YAP protein and/or a nucleotide sequence coding for a wild-type TAZ protein and/or a nucleotide sequence coding for a functional fragment of said wild-type YAP protein and/or said wild-type TAZ protein, and/or a nucleotide sequence coding for said activated version; and/or a nucleotide sequence coding for derivatives thereof.

7. The method according to claim 5, further comprising the step of transfecting said at least one differentiated cell of step a) with a vector comprising a nucleotide sequence coding for a protein which induces the increased expression or activity of said endogenous YAP/TAZ protein.

8. The method according to claim 7, wherein the transfection of said at least one differentiated cell is performed using a lentiviral vector.

9. The method according to claim 8, wherein expression of said wild-type YAP/TAZ protein and/or said functional fragment and or/said activated version, and/or said derivative thereof; or said protein which induces the increased expression or activity of said endogenous YAP/TAZ protein is under the control of an inducible promoter.

10. The method according to claim 9, wherein said inducible promoter is a doxycyclin-inducible promoter.

11. The method according to claim 1, wherein said at least one differentiated cell is a mammalian cell.

12. The method according to claim 1, wherein said at least one differentiated cell is selected of a group of differentiated cells comprising differentiated mammary gland cells, differentiated neural cells and differentiated pancreatic cells.

13. The method according to claim 1, wherein said differentiated cell is a terminal differentiated cell.

14. The method according to claim 1, wherein said step of generating a somatic stem cell comprises verifying at least one characteristic typical for somatic stem cells.

15. A somatic stem cell, obtained by the method according to claim 1.

16. A method of using the somatic stem cell according to claim 15 in a regenerative medicine application.

17. A vector comprising a nucleotide sequence coding for a wild-type YAP protein, and/or a nucleotide sequence coding for a wild-type TAZ protein, and/or a nucleotide sequence coding for a functional fragment of said YAP and/or said TAZ protein, and/or a nucleotide sequence coding for an activated version of said YAP and/or said TAZ protein, and/or a nucleotide sequence coding for a protein which induces an increased expression or activity of an endogenous YAP/TAZ protein, or derivatives thereof, wherein the transcription of said nucleotide sequence is under the control of an inducible promoter, for use in the method of claim 1.

18. The vector of claim 17, wherein the nucleotide sequence comprises anyone of the sequences according to Seq ID No. 1, Seq ID No. 2, Seq ID No. 3, or Seq ID No 4.

19. The vector of claim 18, further comprising the nucleotide sequence according to Seq ID No. 5.

20. A composition comprising a substance for influencing a biological activity of an endogenous YAP/TAZ protein, and/or for influencing a cellular stability of said endogenous YAP/TAZ protein, and/or for influencing a cellular localization of said endogenous YAP/TAZ protein, for use in a method according to claim 1.

21. A kit, comprising the vector of claim 17.

22. The kit according to claim 21, wherein the vector is prepared to be administered orally, rectally, by injection, inhalation, or topically.
